SEQUENCE LISTING (13 sequences)

SEQ ID NO: 1 - protein, 5 aa, Homo sapiens
YIGSR

SEQ ID NO: 2 - protein, 5 aa, Homo sapiens
IKVAV

SEQ ID NO: 3 - protein, 19 aa, Artificial Sequence
  A water-soluble peptide.
CSRARKQAASIKVAVSADR

SEQ ID NO: 4 - protein, 4 aa, Homo sapiens
REDV

SEQ ID NO: 5 - protein, 4 aa, Homo sapiens
RGDS

SEQ ID NO: 6 - protein, 4 aa, Homo sapiens
REDV

SEQ ID NO: 7 - protein, 4 aa, Homo sapiens
RGDV

SEQ ID NO: 8 - protein, 5 aa, Homo sapiens
LRGDN

SEQ ID NO: 9 - protein, 5 aa, Homo sapiens
PDSGR

SEQ ID NO: 10 - protein, 4 aa, Homo sapiens
RGDT

SEQ ID NO: 11 - protein, 4 aa, Homo sapiens
DGEA

SEQ ID NO: 12 - protein, 4 aa, Homo sapiens
  SITE 3: Xaa = any amino acid
VTXG

SEQ ID NO: 13 - protein, 8 aa, Homo sapiens
RNIAEIIK

(19) United States
(12) Patent Application Publication
(10) Pub. No.: US 2010/0291181 A1
(43) Pub. Date: Nov. 18, 2010

(54) THERAPEUTIC DEVICES FOR PATTERNED CELL GROWTH

(75) Inventors: Kathryn E. Uhrich, New Brunswick, NJ (US); Kristine Schmalenberg, New Brunswick, NJ (US)

Correspondence Address: VIKSNINS HARRIS & PADYS PLLP, P.O. BOX 111098, ST. PAUL, MN, 55111-1098, US

(73) Assignee: RUTGERS, THE STATE UNIVERSITY OF NEW JERSEY, NEW BRUNSWICK, NJ (US)

(21) Appl. No.: 12/618,221

(22) Filed: Nov. 13, 2009

Related U.S. Application Data
(63) Continuation of application No. 10/622,072, filed on Jul. 17, 2003, now abandoned.
(60) Provisional application No. 60/396,628, filed on Jul. 17, 2002.

(51) Int. Cl. A61F 2/04; A61K 31/60; A61P 29/00; A61K 38/39

(52) U.S. Cl. 424/426; 514/159; 514/20.9

(57) ABSTRACT

The invention provides therapeutic devices comprising a polymeric anti-inflammatory agent that biodegrades to release anti-inflammatory agents. The therapeutic devices are useful for repair and regeneration of a variety of injured tissues.

### CROSS REFERENCE TO RELATED APPLICATIONS

This application is a continuation of U.S. application Ser. No. 10/622,072 filed on Jul. 17, 2003, and claims priority from U.S. Provisional Patent Application Ser. No. 60/396,628, filed Jul. 17, 2002, which applications are herein incorporated by reference.

### FIELD OF THE INVENTION

The present invention relates to devices that can direct the growth, enhance the regeneration and promote the healing of a variety of tissues, including nerve, bone, muscle, ligament and tendon tissues. The devices comprise polymeric anti-inflammatory agents that facilitate healing of such tissues. Patterns of biologically active molecules can be placed on these devices that guide and encourage growth of selected cell types in selected patterns.

### BACKGROUND OF THE INVENTION

Some types of implantable medical devices have become available to facilitate bone, tooth and tissue repair. Many such devices are made from thermoplastic polymers. Other devices are made from non-absorbable polyamide, aromatic polyester and polyolefin polymers. Still others devices are made from absorbable types of polymers such as poly(lactic acid), poly(glycolic acid), poly(alkylene oxalate), polydioxanone and polyanhydride. See, e.g., U.S. Pat. No. 5,969,020.

A separate field of controlled release devices has developed for sustained or controlled release of therapeutic agents. Many such devices incorporate a therapeutic agent inside a reservoir or within a diffusion-limiting substrate, where the substrate forms a barrier through which the agent must pass in order to enter the patient's biological fluids. Materials that have been used to fabricate diffusion-controlled slow release devices include the non-degradable polymers poly(dimethyl siloxane), ethylene-vinyl acetate copolymers and hydroxylalkyl methacrylates. Cohen et al, U.S. Pat. No. 4,591,496; Folkman et al., U.S. Pat. No. 4,164,560.

In some instances, the controlled release device is used non-invasively. For example, transdermal patches permit sustained delivery of the anti-anginal agent nitroglycerin or scopolamine to prevent motion sickness. Brown, L., and Langer, R. (1988) Ann Rev. Med. 39, 221. In other instances, the controlled release device is implanted. For example, cylindrical nerve guide tubes have been developed that release trophic factors believed to aid nerve regeneration. Aebischer, P. et al. (1989) J. Neurosci. Res. 23, 282.

In any case, it is desirable for implantable delivery devices to slowly degrade in vivo. This obviates the need for, and expense of, an additional surgical procedure to remove the implanted device. Towards that end, diffusion-controlled slow release devices have been fabricated from biodegradable polymers, such as lactic/glycolic acid copolymers (Coombes et al., U.S. Pat. No. 5,290,494; DeLuca et al., U.S. Pat. No. 5,160,745). The geometries of devices that operate by this means include spheres of microscopic and macroscopic dimensions, cylinders, flat sheets, and hollow hemispheres. Langer, R. and Peppas, N. A. (1992) BMES Bull. 16, 3-7 and (1983) J. Macromolec. Sci. 23, 61.

U.S. Pat. Nos. 4,757,128 and 4,997,904 disclose the preparation of polyanhydrides with improved sustained drug release properties from pure, isolated prepolymers of diacids and acetic acid or acetic anhydride. However, these biocompatible and biodegradable aromatic polyanhydrides have aromatic bonds resulting in compounds with slow degradation times as well as relatively insoluble degradation products unless incorporated into a copolymer containing a more hydrophilic monomer, such as sebacic acid. The aromatic polyanhydrides disclosed in the '128 Patent and the '904 Patent are also insoluble in most organic solvents. A bioerodible controlled release device produced as a homogenous polymeric matrix from polyanhydrides with aliphatic bonds having weight average molecular weights greater than 20,000 and an intrinsic velocity greater than 0.3 dL/g and a biologically active substance is also described in U.S. Pat. No. 4,888,176. Another bioerodible matrix material for controlled delivery of bioactive compounds comprising polyanhydride polymers with a uniform distribution of aliphatic and aromatic residues is disclosed in U.S. Pat. No. 4,857,311.

Biocompatible and biodegradable aromatic polyanhydrides prepared from para-substituted bis-aromatic dicarboxylic acids for use on wound closure devices are disclosed in U.S. Pat. No. 5,264,540. However, these compounds exhibit high melt and glass transition temperatures and decreased solubility, thus making them difficult to process. The disclosed polyanhydrides also comprise radical or aliphatic bonds that cannot be hydrolyzed by water.

Polyanhydride polymeric matrices have also been described for use in orthopedic and dental applications. For example, U.S. Pat. No. 4,886,870 discloses a bioerodible article useful for prosthesis and implantation that comprises a biocompatible, hydrophobic polyanhydride matrix. U.S. Pat. No. 5,902,599 also discloses biodegradable polymer networks for use in a variety of dental and orthopedic applications that are formed by polymerizing anhydride prepolymers.

However, while biodegradable medical implants and controlled release devices exist, what is needed is an implantable device that not only can form a scaffold for tissue regeneration but that also releases therapeutically beneficial compounds as it biodegrades.

### SUMMARY OF THE INVENTION

The invention provides a device for tissue regeneration that is made from a polymeric substrate. The polymeric substrate used to make the therapeutic device can be composed of polymers of anti-inflammatory agents. In some embodiments, the polymeric substrate used to make the therapeutic device is coated or co-polymerized with polymers of anti-inflammatory agents. In other embodiments, the polymeric substrate used to make the therapeutic device encapsulates or incorporates polymers of anti-inflammatory agents.

It has unexpectedly been discovered that the local administration of an anti-inflammatory agent to tissue provides beneficial effects on the healing and growth of the tissue, and on proximally located tissues. Biocompatible and biodegradable polymers with polymeric anti-inflammatory agents have therefore been developed with excellent degradation, processing and solubility properties, as well as with therapeutic utilities. As provided herein, these anti-inflammatory polymers can be formed directly into therapeutic devices that are particularly useful in enhancing regeneration and healing of tissues. Alternatively, the anti-inflammatory polymers can be incorporated into therapeutic devises so that the anti-inflammatory agent is slowly released as the device biodegrades.

According to the invention, these anti-inflammatory polymeric devices can be used to treat and repair damaged tissues. Such tissues include any tissue that may require alignment for proper regeneration and/or healing. Examples of such tissues include nervous tissues, muscle tissues, skeletal tissues, ligaments and tendons.

Accordingly, the invention provides a method to promote healing of injured or misaligned tissues comprising implantation of an anti-inflammatory polymeric device at the site of injured or misaligned tissue. The device can be optimally shaped and positioned to facilitate tissue regeneration and to promote proper alignment of the tissue. Factors or biologically active molecules that promote growth and regeneration of the selected tissue or cell type can be incorporated in, on or within the device.

In one embodiment, the anti-inflammatory devices of the invention have a pattern of stably adsorbed or covalently attached biologically active molecules to encourage patterned outgrowth of nerve cells. In vivo implantation of these anti-inflammatory polymeric devices can help the body re-establish nervous connections in damaged or severed peripheral and/or spinal nerves. Accordingly, the invention provides a method to promote healing of nervous tissue comprising implantation of an anti-inflammatory polymeric device at the site of injured nervous tissue. The device can be optimally positioned to facilitate neural regeneration, neurite outgrowth and neural connection to distal tissues.

The devices of the invention are formulated to provide adequate scaffolding for regrowth and regeneration of tissue as well as provide sustained release of an effective amount of an anti-inflammatory agent over a period of at least about 2, about 5, about 10, about 20, or about 40 days. The devices can also be formulated to provide local release of an effective amount of the anti-inflammatory agent over a period of up to about 3 months, about 6 months, about 1 year, or about 2 years.

The devices of the invention can have any shape selected by one of skill in the art. Such a shape is selected to provide optimal support, guidance and reconnection of damaged tissue to healthy tissues.

### BRIEF DESCRIPTIONS OF THE FIGURES

FIG. 1 illustrates a perspective view of a bioactive tubular device that can be formed from the anti-inflammatory polymers of the invention.

FIG. 2 depicts a general method for applying biologically active molecules to the surface of a polymeric substrate without chemically modifying the substrate. The stamp (201) has a pre-selected pattern represented by raised features with particular shapes, in this case the pattern is a series of lines which, when viewed in cross-section, appear to be rectangles projecting from the body of the stamp. The stamp (201) will be used to transfer a pattern of biologically active molecules to the polymeric substrate (202). The polymeric substrate (202) is activated by plasma to generate a transiently polarized surface that can stably bind biologically active molecules. The stamp (201) may also be plasma-treated to facilitate transfer of the biologically active molecules. A heavily coated stamp (203) has been coated with a solution of biologically active molecules (wiggly lines). Excess solution is removed from the heavily coated stamp (203) to generate a coated stamp (204) and the polymeric substrate (202) is stamped, to transfer a pre-selected pattern of biologically active molecules to the polymeric substrate and thereby generate a patterned polymeric surface (205) with the pattern of biologically active molecules.

FIG. 3 is a copy of a photomicrograph illustrating a pattern of biologically active molecules (laminin) stably adsorbed on the surface of a polymeric substrate. In this case the pattern is a series of lines. To permit visualization, the stably adsorbed laminin has been exposed to a solution of fluorescently tagged anti-laminin antibodies. After washing off non-specifically bound antibodies, the bound antibodies were observed under fluorescent illumination.

FIG. 4 is a photomicrograph depicting the pattern of neurite outgrowth from neuronal cells. Neuronal cells were plated onto a pattern of laminin consisting of a series of lines. An optical microscope was used to visualize the pattern of neuronal process outgrowth from the adhered cells. As illustrated, most neuronal processes adhere to and grow along the lines of the laminin pattern.

FIGS. 5A and B provides copies of micrographs of Schwann cells plated onto a pattern of laminin consisting of a series of lines. The image in FIG. 5A was obtained using a Zeiss laser scanning confocal microscope to detect fluorescence at 512 nm. The image in FIG. 5B was obtained using a phase contrast optical microscope. Both images show the pattern of neuronal process outgrowth from cells adhered to a laminin pattern. As illustrated, most neuronal processes adhere to and grow along the lines of the laminin pattern.

FIG. 6 is a micrograph taken randomly of fluorescently stained Schwann cells. The fluorescent images of the stained Schwann cells were converted to black/white, then thresholded to separate the cell images.

FIG. 7 provides a graph showing the average orientation of the monolayer of Schwann cells plated on a pattern of laminin lines as shown in FIG. 6. The direction of laminin patterning was represented by 0° and the frequencies were normalized and plotted. Cells and aggregates like those shown in FIG. 6 were fitted with ellipses. Major and minor axes were noted as well as the angle created by the major axis relative to the direction of laminin patterning. The frequency at which the orientation angle of each major elliptical axis differed from 0° was noted and these frequencies were separated into 10° increments from 0-180°. The mean angle was determined and converted to 0°, with a new range of +90°, so that distributions could be compared. As illustrated, the majority of the cells are oriented at 0°, in other words, in the same orientation as the lines of laminin.

### DETAILED DESCRIPTION OF THE INVENTION

According to the invention, tissue growth, regeneration and/or alignment can be enhanced by use of polymeric anti-inflammatory agents. Devices formed or containing such polymeric anti-inflammatory agents enhance tissue growth by providing scaffold that appropriately guides the growth of tissues as well as a slowly degrading anti-inflammatory that enhances healing and growth of the tissues. Moreover, according to the invention, the interior and/or exterior of the therapeutic devices can have a pattern of adsorbed or covalently attached biologically active molecules to further encourage the growth of selected cell types in a selected pattern.

Applicant has previously observed that local administration of a polymeric anti-inflammatory agent enhances the growth and regeneration of tissues. Polymeric anti-inflammatory agents are biodegradable and provide a controlled release of the agent at or near the implantation site over a period of days or months. Such controlled release of anti-inflammatory agents encourages tissue repair and regeneration.

However, in many cases, specific tissue connections must be re-established in order for crushed or severed tissues to be effectively repaired. The devices of the invention facilitate formation of appropriate tissue connections by providing polymeric anti-inflammatory agents in a variety of forms upon and along which tissues and cellular processes are guided and encouraged to grow. The devices of the invention further provide biologically active molecules that are stably adsorbed onto or covalently attached to the device in selected patterns. Alternatively, the biologically active molecules incorporated into the interior of the devices. Such biologically active molecules further stimulate cell growth and augment the establishment of new tissue connections. Hence, the devices of the invention facilitate tissue growth and regeneration in several ways.

### DEFINITIONS

As used herein the term, “sustained release” means that the agent is formulated such that it will be released over an extended period of time when administered according to the methods of the invention. For example, the agent can conveniently be formulated so that it will be released over a period of at least about 2, about 5, about 10, about 20, or about 40 days. Preferably, the agent is formulated so that it is released over at least about 5 or about 10 days. The agent can also preferably be formulated so that it is released over a period of about 30 to about 90 days. For the treatment of damaged tissue, the agent is preferably formulated so that it is released over a period of about 1 to about 30 days, more preferably about 2 to about 25 days.

As used herein, an agent is “appended” to a polymer when the agent is bonded to the polymer as a side chain or side group, but is not part of the polymer backbone. Preferably, the agent is bonded to the polymer through a linkage that is suitable to release the agent when the polymer is administered according to the methods of the invention. For example, the agent can conveniently be linked to a polymer through a hydrolyzable linkage such as an anhydride or ester linkage.

As used herein, the term “dispersed through the polymer matrix” means that an anti-inflammatory agent is located within the matrix of a polymer such that it can be released in a controlled fashion within the body. Preferably, the polymer matrix comprises a biodegradable polymer.

As used herein, the term “healing” means the restoration of injured or damaged tissue to at least minimal function.

### Therapeutic Devices

The shapes and sizes of devices of the invention can vary to suit any application desired by one of skill in the art. In particular, the shape and size of a device can be selected to optimally treat the site of an injured, severed or crushed tissue, for example, any injured, severed or crushed muscle, ligament, bone, tendon, nerve or a combination thereof. Such shapes and sizes are selected to permit growth of the selected tissue within or along the device so that the function of the tissue can be improved at or distal to the site of the injured, severed or crushed tissue.

Shapes contemplated for the devices of the invention include tubes, “jelly rolls,” rods, sheets, fibers, meshes, and the like, as well as irregularly shaped devices that are designed to be adapted to fit or fill a specific anatomical site or the site of a specific injury. Devices of the invention can have protuberances, indentations, crevices, pores or tubes running through them so that the device can optimally adapt to an implantation site and/or so that tissue regeneration is optimized within or on the device.

In one embodiment, the device is a “jellyroll” comprising a biodegradable, polymeric sheet that is rolled into a tubular device. Such a jellyroll device permits tissues, cells, neuronal processes and the like to grow along and between the layers of the rolled sheet. The sheet can have ridges or spacers to help separate the rolled layers of the sheet so that tissues, cells and neuronal processes can more easily grow between those layers. Such ridges or spacers can be configured to optimally guide the growth and extension of tissues such as neural processes. For example, the sheet can have multiple linear ridges that run the length of the sheet so that upon rolling, the rolled sheet forms a porous tube with a multitude of tubules running from one end of the jellyroll to the other. The height of the ridges or spacers is selected so that, upon rolling up the sheet, the tubules formed will be able to optimally accommodate growing tissues such as neuronal processes. Hence, the ridges or spacers can be, for example, about one micron to about fifty microns, or about two microns to about thirty microns, or about three microns to about ten microns, or of another height selected by one of skill in the art.

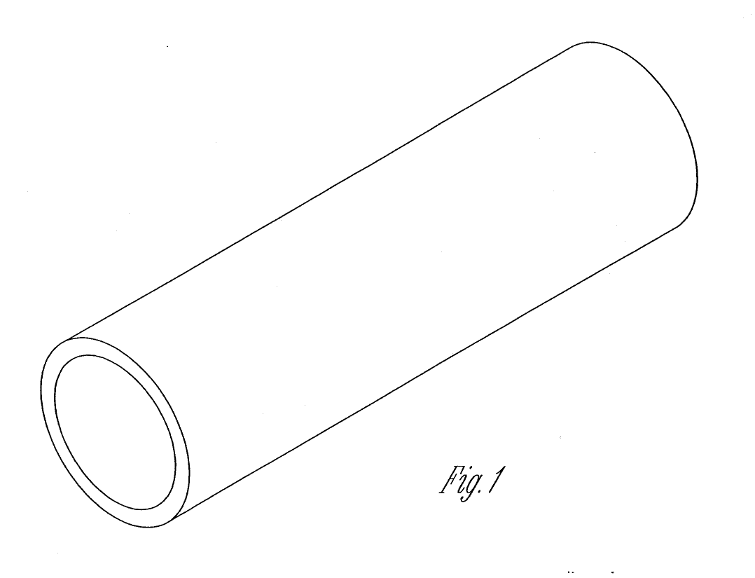

In another embodiment, the device is a tube that is of the approximate diameter to accommodate an uninjured tissue (such as a muscle, bone, ligament, tendon or nerve, see FIG. 1). In particular, the diameter of such a tubular device may be estimated from the diameter of an uninjured segment of the injured, severed or crushed tissue. Diameters contemplated for the tubular devices of the invention include diameters ranging from about one millimeter to about ten centimeters, preferably about three millimeters to about five centimeters and more preferably about five millimeters to about two centimeters.

The length of such tubular or “jellyroll” devices is generally selected to span the region of injury so that the regenerating tissue can form connections with uninjured tissues on either side of the site of injury. Lengths contemplated include lengths ranging from about one millimeter to about twenty centimeters, or about three millimeters to about ten centimeters or about five millimeters to about five centimeters.

In some instances it may be desirable to make the devices of the invention somewhat porous, particularly when the devices are somewhat large, long or wide. When this is desirable, the devices can be made porous by providing holes in the polymeric substrate of device.

The devices of the invention generally comprise a biodegradable polymeric substrate comprising at least one type anti-inflammatory agent. One or more interior or exterior surfaces of the devices can also have a pattern of biologically active molecules that provide topographical and chemical cues that encourage cell attachment and growth. These aspects of the invention are described in more detail below.

### Anti-Inflammatory Agents

Anti-Inflammatory agents are a well-known class of pharmaceutical agents that reduce inflammation by acting on body mechanisms (Stedman's Medical Dictionary 26 ed., Williams and Wilkins, (1995); Physicians Desk Reference 51 ed., Medical Economics, (1997)).

Anti-inflammatory agents useful in the devices of the invention include Non-Steroidal Anti-Inflammatory Agents (NSAIDS), such as those described in U.S. application Ser. No. 09/732,516. NSAIDS typically inhibit the body's ability to synthesize prostaglandins. Prostaglandins are a family of hormone-like chemicals, some of which are made in response to cell injury. Specific NSAIDS approved for administration to humans include aspirin, naproxen sodium, diclofenac, sulindac, oxaprozin, diflunisal, piroxicam, indomethocin, etodolac, ibuprofen, fenoprofen, ketoprofen, mefenamic acid, nabumetone, tolmetin sodium, and ketorolac tromethamine. Other anti-inflammatory agents useful in the methods of the invention include salicylates, such as, for example, salicylic acid, acetyl salicylic acid, choline salicylate, magnesium salicylate, sodium salicylate, olsalazine, and salsalate.

Other anti-inflammatory agents useful in the methods of the invention include cyclooxygenase (COX) inhibitors. COX catalyzes the conversion of arachidonate to prostaglandin H2 (PGH2); a COX inhibitor inhibits this reaction. COX is also known as prostaglandin H synthase, or PGH synthase. Two Cox genes, Cox-1 and Cox-2 have been isolated in several species. COX-2 is tightly regulated in most tissues and usually only induced in abnormal conditions, such as inflammation, rheumatic and osteo-arthritis, kidney disease and osteoporosis. Cox-1 is believed to be constitutively expressed so as to maintain platelet and kidney function and integral homeostasis. Typical COX inhibitors useful in the methods of the invention include etodolac, celebrex, meloxicam, piroxicam, nimesulide, nabumetone, and rofecoxib.

Anti-inflammatory agents that can be admixed into a polymer matrix for the devices of the invention include, for example: Isonixin, Amtolmetin Guacil, Proglumetacin, Piketoprofen, Difenamizole, Epirizole, Apazone, Feprazone, Morazone, Phenylbutazone, Pipebuzone, Propyphenazone, Ramifenazone, Thiazolinobutazone, Benorylate, Calcium Acetylsalicylate, Etersalate, Imidazole Salicylate, Lysine Acetylsalicylate, Morpholine Salicylate, 1-Naphthyl Salicylate, Phenyl Acetylsalicylate, Ampiroxicam, Droxicam, Amixetrine, Benzydamine, Bucolome, Difenpiramide, Emorfazone, Guaiazulene, Nabumetone, Nimesulide, Proquazone, and Superoxide Dismutase.

Anti-inflammatory agents that can be appended to a polymer for administration in the methods of the invention include, for example: Etofenamate, Talniflumate, Terofenamate, Acemetacin, Alclofenac, Bufexamac, Cinmetacin, Clopirac, Felbinac, Fenclozic Acid, Fentiazac, Ibufenac, Indomethacin, Isofezolac, Isoxepac, Lonazolac, Metiazinic Acid, Mofezolac, Oxametacine, Pirazolac, Sulindac, Tiaramide, Tolmetin, Tropesin, Zomepirac, Bumadizon, Butibufen, Fenbufen, Xenbucin, Clidanac, Ketorolac, Tinoridine, Benoxaprofen, Bermoprofen, Bucloxic Acid, Fenoprofen, Flunoxaprofen, Flurbiprofen, Ibuprofen, Ibuproxam, Indoprofen, Ketoprofen, Loxoprofen, Naproxen, Oxaprozin, Pirprofen, Pranoprofen, Protizinic Acid, Suprofen, Tenidap, Tiaprofenic Acid, Zaltoprofen, Benzpiperylon, Mofebu atone, Oxyphenbutazone, Suxibuzone, Acetaminosalol, Parsalmide, Phenyl Salicylate, Salacetamide, Salicylsulfuric Acid, Isoxicam, Lomoxicam, Piroxicam, Tenoxicam, ε-Acetamidocaproic Acid, Bendazac, α-Bisabolol, Paranyline, and Perisoxal.

Anti-inflammatory agents that can be incorporated into a polymer backbone for administration in the methods of the invention include, for example: Aceclofenac, Alminoprofen, 3-Amino-4-hydroxybutyric Acid, Bromfenac, Bumadizon, Carprofen, 5-Chlorosalicylic acid, Diclofenac, Diflunisal, Ditazol, Enfenamic Acid, Etodolac, Fepradinol, Flufenamic acid, Glucametacin, Meclofenamic acid, Mefenamic acid, Niflumic acid, Oxaceprol, S-Adenosylmethionine, Salsalate, Tolfenamic acid, 5-Trifluoromethylsalicylic acid, Ximoprofen and Zileuton.

For any anti-inflammatory agent referred to herein by a trade name it is to be understood that either the trade name product or the active ingredient possessing anti-inflammatory activity from the product can be used. Additionally, preferred agents identified herein for incorporation into a polymer backbone can also preferably be appended to a polymer or can be incorporated into a polymer matrix. Preferred agents that can be appended to a polymer can also preferably be incorporated into a polymer matrix.

Polymers for Use with the Invention

Any anti-inflammatory agent may be used in the polymeric substrate that forms a device of the invention. The polymers used to form the anti-inflammatory devices of the invention may be prepared in accordance with methods commonly employed in the field of synthetic polymers. Many anti-inflammatory agents can be polymerized to form a polymeric substrate or appended to a polymeric substrate and thereby used in the devices of the invention by intermolecular reaction of available functional groups on the selected anti-inflammatory agent. However, the ability of an anti-inflammatory agent to be incorporated into or appended to a polymer substrate may depend on the functional groups present in the agent. In an alternative embodiment, any anti-inflammatory agent can be dispersed through and trapped within a polymer matrix to provide a suitable polymeric substrate. The invention provides homopolymers that can be prepared from suitably functionalized anti-inflammatory agent. However, the mechanical and hydrolytic properties of polymers comprising one or more anti-inflammatory agents can be controlled by incorporating a linking group (L) into the polymer backbone. Accordingly, in one embodiment the polymers of the invention comprise backbones wherein anti-inflammatory agents and linker groups are bonded together through ester linkages, thioester linkages, amide linkages, thioamide linkages, anhydride linkages or a mixture thereof. Due to the presence of the ester, thioester, amide, thioamide, anhydride and/or linkages, the polymers used to make the therapeutic devices of the invention can be hydrolyzed under physiological conditions to release the individual anti-inflammatory agent(s). Thus, the polymers used to make the devices of the invention are biodegradable, and in the process of biodegradation, the devices release useful anti-inflammatory agents in a controlled fashion that helps to heal and regenerate tissues at the site of implantation.

Anti-inflammatory agents that can be incorporated into the polymers of the invention possess at least two functional groups that can each be incorporated into an ester, thioester, or amide linkage of a polymer, such that, upon hydrolysis of the polymer, the anti-inflammatory agent is released. These groups can independently be hydroxy groups (—OH), a mercapto groups (—SH), amine groups (—NHR), or carboxylic acids (—COOH). The anti-inflammatory agents can also comprise other functional groups that are not necessarily employed in the formation of the polymer but that can be used to modify the properties of the polymer, including hydroxy groups, mercapto groups, amine groups, carboxylic acids, and the like. Such functional groups can be used or involved in branching, cross-linking, appending other molecules (e.g. biologically active molecules) to the polymer, changing the solubility of the polymer, or affecting the biodistribution of the polymer.

A specific polymer of the invention comprises one or more units of formula I:

—R₁-A-L-A-R₁ I

wherein R₁ is group that will provide an anti-inflammatory agent upon hydrolysis of the polymer; each A is independently an amide linkage, a thioester linkage, or an ester linkage; and L is a linking group.

For example, the following illustrates the hydrolytic degradation of poly(anhydride-ester) (1) to form salicyclic acid (2) and sebacic acid (3).

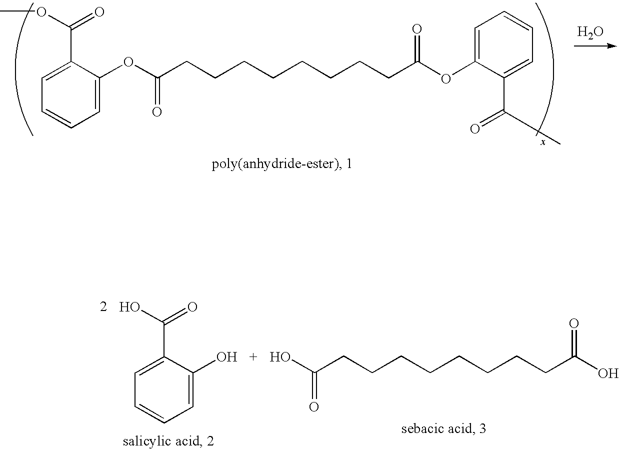

Another specific polymer of the invention is a polymer that comprises one or more units of formula II:

—R₂-A-L-A-R₂-R₃-A-L-A-R₃— II

wherein: R₂ and R₃ are each independently a group that will yield an anti-inflammatory agent upon hydrolysis of the polymer; each A is independently an amide, thioester, thioamide or ester linkage; and each L is independently a linking group.

As provided above, the R₁, R₂ and R₃ groups of the invention will yield an anti-inflammatory agent upon hydrolysis of the polymer. Such anti-inflammatory agents include aceclofenac, acemetacin, ε-acetamidocaproic acid, acetaminosalol, alclofenac, alminoprofen, 3-amino-4-hydroxybutyric acid, amixetrine, ampiroxicam, amtolmetin guacil, apazone, bendazac, benorylate, benoxaprofen, benzpiperylon, benzydamine, bermoprofen, α-bisabolol, bucolome, bucloxic acid, bufexamac, bumadizon, butibufen, calcium acetylsalicylate, carprofen, choline salicylate, cinmetacin, clopirac, clidanac, diclofenac, difenamizole, difenpiramide, diflunisal, ditazol, droxicam, emorfazone, enfenamic acid, epirizole, etersalate, etodolac, etofenamate, felbinac, fenbufen, fenclozic acid, fenoprofen, fentiazac, fepradinol, feprazone, flunoxaprofen, flurbiprofen, glucametacin, guaiazulene, ibufenac, ibuprofen, ibuproxam, imidazole salicylate, indomethacin, indoprofen, isofezolac, isonixin, isoxepac, isoxicam, ketoprofen, ketorolac, lomoxicam, lonazolac, loxoprofen, lysine acetylsalicylate, mefenamic acid, metiazinic acid, mofebutazone, mofezolac, morazone, morpholine salicylate, nabumetone, 1-naphthyl salicylate, naproxen, nimesulide, oxaceprol, oxametacine, oxaprozin, oxyphenbutazone, paranyline, parsalmide, perisoxal, phenyl acetylsalicylate phenylbu alone, phenyl salicylate, piroxicam, piketoprofen, pipebuzone, pirazolac, piroxicam, pirprofen, pranoprofen, proglumetacin, propyphenazone, proquazone, protizinic acid, ramifenazone, S-adenosylmethionine, salacetamide, salsalate, salicylic acid, salicylsulfuric acid, sulindac, superoxide dismutase, suprofen, suxibuzone, talniflumate, tenidap, tenoxicam, terofenamate, thiazolinobutazone, tiaprofenic acid, tiaramide, tinoridine, tropesin, xenbucin, ximoprofen, zaltoprofen, zileuton, zomepirac, and the like.

Many such R₁, R₂ and R₃ groups are substituted or unsubstituted alkylaryl groups or aromatic rings. Such aromatic rings can contain five to fourteen ring carbons. Aromatic rings contemplated by the invention include phenyl, naphthyl and related rings. Preferably, the R₁, R₂ and R₃ groups are substituted phenyl rings containing at least one, preferably two, free alcohols, aldehyde, amine, carbonyl, carboxylate, halo, thiol or other reactive groups available for polymerization. Such groups facilitate co-polymerization with reactive groups on the linker (L).

The nature of the linking group “L” in a polymer of the invention is not critical provided the polymer of the invention possesses acceptable mechanical properties and release kinetics for the devices of the invention to promote healing and regeneration of neural tissues. The linking group L is typically a divalent organic radical having a molecular weight of from about 25 daltons to about 400 daltons. More preferably, L has a molecular weight of from about 40 daltons to about 200 daltons.

The linking group L typically has a length of from about 5 angstroms to about 100 angstroms using standard bond lengths and angles. More preferably, the linking group L has a length of from about 10 angstroms to about 50 angstroms.

The linking group L may be biologically inactive, or may itself possess biological activity. The linking group can also comprise other functional groups (including hydroxy groups, mercapto groups, amine groups, carboxylic acids, as well as others) that can be used to modify the properties of the polymer (e.g. for branching, for cross linking, for appending other molecules, such as the biologically active molecules described herein, to the polymer, for changing the solubility of the polymer, or for effecting the biodistribution of the polymer). L can also be an amino acid or a peptide.

In one embodiment, L is a divalent, branched or unbranched, saturated or unsaturated, hydrocarbon chain, having from 1 to 25 carbon atoms, wherein one or more (e.g. 1, 2, 3, or 4) of the carbon atoms are optionally replaced by (—O—) or (—NR—). One or more carbon atom of the hydrocarbon chain can optionally be substituted with one or more (e.g. 1, 2, 3, or 4) substituents selected from the group consisting of (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo (═O), carboxy, aryl, aryloxy, heteroaryl, and heteroaryloxy.

Preferably, L is a divalent. A preferred length for the hydrocarbon chain of L is from 3 to 15. A more preferred length for the hydrocarbon chain of L is 6 to 10 carbon atoms. An even more preferred length for the hydrocarbon chain of L is a divalent hydrocarbon chain having 7, 8, or 9 carbon atoms. A most preferred length for the hydrocarbon chain of L is a divalent hydrocarbon chain having 8 carbon atoms.

The polymeric substrate of the inventive devices can be formed by available procedures. For example, a polymer of the invention can be prepared, as illustrated in Scheme A, from an anti-inflammatory agent of formula (X₁—R₁—X₂) and a linker precursor of formula Z₁-L-Z₂, wherein X₁, X₂, Z₁, and Z₂ are selected from the values in Table 1 below.

X₁—R₁—X₂+Z₁-L-Z₂→—(R₁-A-L-A)ₙ- A

Depending on the reactive functional group (X₁ or X₂) of the anti-inflammatory compound, a corresponding L functional group (Z₁ or Z₂) can be selected from Table 1, to provide an ester linkage, thioester linkage, amide linkage or thioamide linkage in the polymer backbone.

TABLE 1

| Functional Group on an | Functional Group On | Resulting |
|---|---|---|
| Anti-inflammatory Agent | Linker Precursor | Linkage |
| (X₁ or X₂) | (Z₁ or Z₂) | In Polymer (A) |
| —COOH | —OH | Ester |
| —COOH | —NHR | Amide |
| —COOH | —SH | Thioester |
| —OH | —COOH | Ester |
| —SH | —COOH | Thioester |
| —NHR | —COOH | Amide |
| —NHR | —C(S)OH | Thioamide |

The anti-inflammatory agent and the linker precursor can be polymerized using available synthetic techniques (e.g. by condensation) to provide a polymer of the invention (e.g. I or II) wherein each A is independently an ester linkage, a thioester linkage, an amide linkage, a thioamide linkage or an anhydride linkage.

As will be clear to one skilled in the art, suitable protecting groups can be used during the reactions illustrated in Scheme A (and in the reactions illustrated in the Schemes below). For example, other functional groups present in the biologically active compound or the linker precursor can be protected during polymerization, and the protecting groups can subsequently be removed to provide the polymer of the invention. Suitable protecting groups and methods for their incorporation and removal are well known in the art (see for example Greene, T. W.; Wutz, P. G. M. “Protecting Groups In Organic Synthesis” second edition, 1991, New York, John Wiley & sons, Inc.).

Additionally, when a carboxylic acid is reacted with a hydroxy group, a mercapto group, or an amine group to provide an ester linkage, thioester linkage, or an amide linkage, the carboxylic acid can be activated prior to the reaction, for example, by formation of the corresponding acid chloride. Numerous methods for activating carboxylic acids, and for preparing ester linkages, thioester linkages, and amide linkages, are known in the art (see for example Advanced Organic Chemistry: Reaction Mechanisms and Structure, 4 ed., Jerry March, John Wiley & Sons, pages 419-437 and 1281).

A polyester of the invention can be formed from an anti-inflammatory compound of formula (HO—R₁—OH) and a linker precursor of formula HOOC-L-COOH, as illustrated in Scheme B.

HO—R₁—OH+HOOC-L-COOH→(R₁—O—CO-L-CO—O)ₙ— B

Reaction of the hydroxy groups of the anti-inflammatory compound (HO—R₁—OH) with the carboxylic acids of the linker precursor (HOOC-L-COOH) provides a polymer of formula III, which is a polymer of the invention:

—(R₁—O—CO-L-OC—O)ₙ— III

Accordingly, dihydroxy anti-inflammatory agents can be polymerized to prepare a polyester substrate useful for preparation of the devices of the invention.

A polyamide of the invention can be prepared using a procedure similar to that illustrated in Scheme B by replacing the dihydroxy anti-inflammatory compound in Scheme B with a suitable diamino anti-inflammatory compound.

A polythioester of the invention can be prepared using a procedure similar to that illustrated in Scheme B by replacing the dihydroxy anti-inflammatory compound in Scheme B with a suitable dimercapto anti-inflammatory compound.

A polyester/polyamide of the invention can be formed from an anti-inflammatory compound of formula (HRN—R₁—OH) and from a linker precursor of formula HOOC-L-COOH as illustrated in Scheme C.

HRN—R₁—OH+HOOC-L-COOH→—(R₁—O—CO-L-CO—NR)ₙ— C

Reaction of the hydroxy group and the amino group of the anti-inflammatory agent with the carboxylic acids of the linker precursor provides a polymer having formula IV [—(R₁—O—CO-L-CO—NR)ₙ—], which is a polymer of the invention.

A polythioester/polyamide of the invention can be prepared using a procedure similar to that illustrated in Scheme C by replacing the hydroxy/amino anti-inflammatory agent in Scheme C with a suitable mercapto/amino anti-inflammatory agent.

A polyamide of the invention can be formed from an anti-inflammatory agent of formula (HOOC—R₁—COOH) and from a linker precursor of formula HRN-L-NRH as illustrated in Scheme D.

HOOC—R₁—COOH+HRN-L-NRH→—(R₁—CO—NR-L-NR—CO)ₙ D

Reaction of the carboxylic acid groups of anti-inflammatory agents with the amino groups of the linker precursor provides a polymer of formula V [—(R₁—CO—NR-L-NR—CO)ₙ], which is a polymer of the invention.

A polyester substrate for the devices of the invention can be prepared using a procedure similar to that illustrated in Scheme D by replacing the diamino linker precursor with a dihydroxy linker precursor. Similarly, a polyester/polyamide substrate for the devices of the invention can be prepared using a procedure similar to that illustrated in Scheme D by replacing the diamino linker precursor with an hydroxy/amino linker precursor. A polythioester/polyamide substrate for the devices of the invention can be prepared using a procedure similar to that illustrated in Scheme D by replacing the diamino linker precursor with a mercapto/amino linker precursor.

A polyester substrate for the devices of the invention can also be formed from an anti-inflammatory agent of formula (HO—R₁—COOH) and from a linker precursor of formula HO-L-COOH as illustrated in Scheme E.

HO—R₁—COOH+HO-L-COOH→—(R₁—CO—O-L-CO—O)ₙ— E

Reaction of the hydroxy group and the carboxylic acid of the anti-inflammatory compound with the carboxylic acid and the hydroxy group of the linker precursor provides a polymer of formula VI [—(R₁—CO-L-OC—O)ₙ—], which is a polymer of the invention.

A polyester/polyamide of the invention can be prepared using a procedure similar to that illustrated in Scheme E by replacing hydroxy/carboxylic anti-inflammatory agents with an amino/carboxylic acid anti-inflammatory compound. A polythioester/polyester of the invention can be prepared using a procedure similar to that illustrated in Scheme E by replacing the hydroxy/carboxylic anti-inflammatory agent with a mercapto/carboxylic acid anti-inflammatory compound.

The preparation of a polymer of the invention comprising 5-aminosalicylic acid is illustrated in Scheme F.

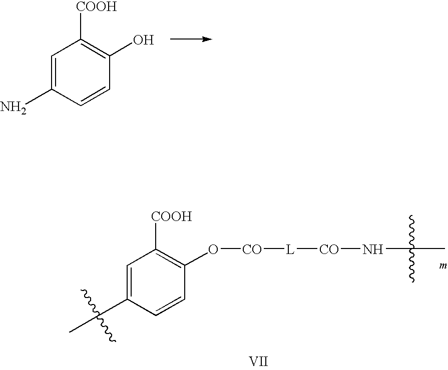

The linking group L utilized in Scheme F is preferably —(CH₂)ₓ—, and more preferably, L is —(CH₂)₈—. The linker precursor employed can have reactive carbonyl functional groups that contribute to the formation of the ester linkage between the L and 5-aminosalicilic acid. For example, the linker precursor can be of the formula HOOC-L-COOH, or a related compound.

A polymer of formula VII, wherein L is as described herein, is a preferred polymer of the invention. For a polymer of formula VII, m is an integer that is greater than or equal to 2. Prior to the polymerization illustrated in Scheme F, the carboxylic acid can be protected with a suitable protecting group, which can subsequently be removed, to provide the polymer of the invention.

5-Aminosalicilic acid can also be incorporated into a polymer of the invention that has formula VIII or IX:

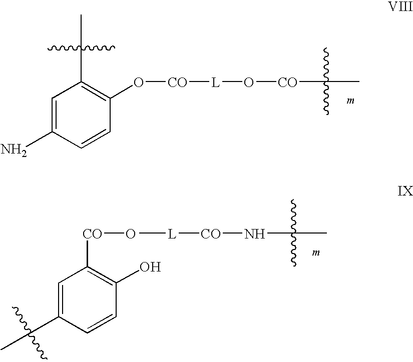

In another embodiment, anti-inflammatory agents can be appended to or incorporated into aromatic polyanhydride polymers. Such anti-inflammatory polyanhydride polymers can be made according to Scheme G.

2X₁—R₁—X₂+Z₁-L-Z₂→—(CO—R₁-A-L-R₁—CO—O)ₙ— G

wherein A is independently an ester linkage, a thioester linkage, or an amide linkage; X₁ and X₂ are reactive functional groups on the anti-inflammatory compound, and Z₁ and Z₂ are functional groups on the linker (L). To form the anhydride of formula X [—(CO—R₁-A-L-A-R₁—CO—O)ₙ—], one of the X₁ or X₂ groups is a carboxylic acid group.

In one preferred embodiment, the polyanhydride anti-inflammatory is a polymer of formula XI:

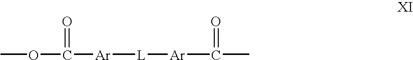

wherein Ar is a substituted or unsubstituted aromatic ring and L is a linker as provided herein.

The invention also provides a dimeric anhydride of formula XII:

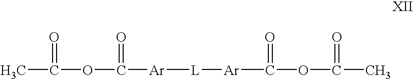

wherein Ar and L are as described above. One example of a compound falling within formula XII is a compound of formula XIII.

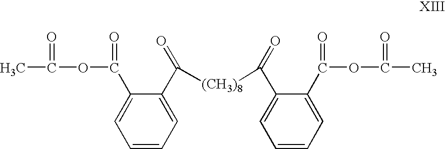

Aromatic rings contemplated for Ar include phenyl and naphthyl. Upon biodegradation of a polymer having any of formulae X-XIII, anti-inflammatory agents are formed. Hence, Ar is related to R₁ in that it contributes to the formation of anti-inflammatory agents upon degradation, but Ar constitutes a smaller set of moieties, namely those that contain substituted or unsubstituted aromatic rings. Preferably, Ar is a divalent substituted phenyl. More preferably, Ar is an ortho-substituted divalent phenyl.

While L can be any of the groups listed for L, the preferred L groups for compounds of formula X-XIII are divalent, branched or unbranched, saturated or unsaturated, hydrocarbon chains having from 1 to 25 carbon atoms. A preferred length for the hydrocarbon chain of L is from 3 to 15 carbon atoms. A more preferred length for L hydrocarbon chains is 6 to 10 carbon atoms. An even more preferred length for the hydrocarbon chain of L is 7, 8, or 9 carbon atoms. A most preferred length for the hydrocarbon chain of L is 8 carbon atoms.

The polyanhydrides of the present invention may be prepared by the method described in Conix, Macromol. Synth., 2, 95-99 (1996), in which dicarboxylic acids are acetylated in an excess of acetic anhydride followed by melt condensation of the resulting carboxylic acid anhydride at 180° C. for 2-3 hours. The resulting polymers are isolated by precipitation into diethyl ether from methylene chloride. The described process is essentially the conventional method for polymerizing bis-aromatic dicarboxylic acid anhydrides into aromatic polyanhydrides.

Polyanhydrides of formulae X-XIII have excellent degradation properties and processability. The preparation of aromatic polyanhydrides from ortho-substituted bis-aromatic carboxylic acid anhydrides disrupts the crystallinity of the resulting polymer, enhancing solubility and processability, as well as degradation properties. The use of hydrolyzable bonds such as esters, amides, urethanes, carbamates and carbonates as opposed to aliphatic bonds in these compounds further enhances these properties.

Polymers of anti-inflammatory agents used to make the devices of the invention have average molecular weights of about 1500 daltons, up to about 300,000 daltons, calculated by Gel Permeation Chromatography (GPC) relative to narrow molecular weight polystyrene standards. Preferred polymers have average molecular weights of about 10,000 daltons, up to about 200,000 daltons, or about 30,000 daltons to about 100,000 daltons.

Polymers of formulae I, and III-XIII illustrated in Schemes A-G above, R₁, A, L, and R can have any of the values, specific values, or preferred values described herein.

The R₁, A, L and R groups are preferably selected so that the hydrolysis products of the polymers of the invention have a chemical structure and activity resembling an anti-inflammatory agent. Preferred hydrolysis products have chemical structures and activities resembling salicylates such as aspirin, non-steroidal anti-inflammatory naphthyl or phenyl propionates such as ibuprofen, ketoprofen, naproxen, and the like, or other aromatic anti-inflammatory compounds such as indomethacin, indoprofen, and the like. Preferred anti-inflammatory agents that posses suitable ortho functional groups to be incorporated into the backbone of a polymer of any one of formula I-XIII as described herein include: Flufenamic Acid, Meclofenamic Acid, Mefenamic Acid, Niflumic Acid, Tolfenamic Acid, Amfenac, Bromfenac, Diclofenac Sodium, Etodolac, Bromosaligenin, Diflunisal, Fendosal, Gentisic Acid, Glycol Salicylate, Salicilic Acid, Mesalamine, Olsalazine, Salicylamide O-Acetic Acid, Sulfasalazine, and the like. Examples of the therapeutically useful salicylates include, but are not limited to, thymotic acid, 4,4-sulfinyldinailine, 4-sulfanilamidosalicylic acid, sulfanilic acid, sulfanilylbenzylamine, sulfaloxic acid, succisulfone, salicylsulfuric acid, salsallate, salicylic alcohol, salicylic acid, orthocaine, mesalamine, gentisic acid, enfenamic acid, cresotic acid, aminosalicylic acid, aminophenylacetic acid, acetylsalicylic acid, and the like. Preferred salicylates for incorporation into the polymeric anti-inflammatory substrates of the invention are salicylic acid, thymotic acid, 4-sulfanilamidosalicylic acid, mesalamine, gentisic acid, enfenamic acid, cresotic acid, or aminosalicylic acid.

The polymeric anti-inflammatory agents used in the devices of the invention can be isolated by known methods commonly employed in the field of synthetic polymers. The polymers can be readily processed into pastes and gels that solidify after being shaped into the device. Alternatively, the polymers can be solvent cast to yield various shapes of various sizes for medical implants. The polymeric anti-inflammatory agents of the invention may also be processed into differently shaped devices by compression molding and extrusion.

### Biologically Active Molecules

Biologically active molecules can create topographical and chemical cues that have been used successfully to control cell attachment. Britland et al., Experimental Cell Research 1992, 198, 124-129; DeFife et al., Journal of Biomedical Materials Research 1999, 45, 148-154; Folch et al., Biotechnology Progress 1998, 14, 388-392; Fraser, S. E. Developmental Biology 1980, 79, 453-464. Such topographical and chemical cues have also been used successfully to control cell spreading. Brunette, D. M. Experimental Cell Research 1986, 164, 11-26; Brunette et al., B. Journal of Biomechanical Engineering 1999, 121, 49-57; Chen, C. S.; Mrksich et al., Biotechnology Progress 1998, 14, 356-363; Clark et al., Journal of Cell Science 1991, 99, 73-77; Craighead et al., Journal of Biomedical Microdevices 1998, 1, 49-64; Curtis et al., Critical Reviews in Biocompatibility 1990, 5, 343-362. In addition, topographical and chemical cues have been used successfully to control cell growth. Carter, S. B. Nature 1965, 208, 1183-1187; Chen et al., Science 1997, 276, 1425-28; Hammarback et al., Developmental Biology 1986, 117, 655-662.

The term “biologically active molecule” is used herein to denote a molecule that can be covalently attached to, or stably adsorbed onto, the surface of the polymeric substrate of the present inventive devices and that has a useful in vivo or in vitro function. Biologically active molecules therefore include any molecule that can affect a biological process, such as cellular adhesion, growth or differentiation. Biologically active molecules can be peptides, proteins, carbohydrates, nucleic acids, lipids, polysaccharides, proteoglycans, synthetic inorganic or organic molecules or combinations thereof. Lists of biologically active molecules can be found, for example, in: Physicians Desk Reference, 55 ed., 2001, Medical Economics Company, Inc., Montvale N.J.; USPN Dictionary of USAN and International Drug Names, 2000, The United States Pharmacopeial Convention, Inc., Rockville, Md.; and The Merck Index, 12 ed., 1996, Merck & Co., Inc., Whitehouse Station, N.J. One skilled in the art can readily select biologically active molecules that possess the necessary functional groups for incorporation into the polymers of the invention from such lists.

The following biologically active molecules are understood to be exemplary and are not to be limiting in any manner. Examples of biologically active molecules for use in the present invention include hormones, extracellular matrix molecules, cell adhesion molecules, natural polymers, enzymes, peptides, antibodies, antigens, polynucleotides, growth factors, synthetic polymers, polylysine, drugs and other molecules. For example, biologically active molecules such as bioadhesive peptides, polylysine, fibronectin, collagen, polyethylene glycol and thrombospondin can be used in the invention.

In one embodiment, biologically active molecules that promote the adhesion, growth and/or differentiation of specific cell types (e.g. neural cells) are used. Examples of biologically active molecules that also may be used include cellular binding domains of extracellular matrix proteins and other adhesion proteins, for example fibronectin and vitronectin, or fragments thereof, that are recognized by cytoskeleton associated receptors in the cell membrane, known as integrins.

For example, laminin, a protein found in the extracellular matrix, enhances neuronal and Schwann cell attachment and migration. David et al., Journal of Neuroscience Research 1995, 42, 594-602; Son et al., Neuron 1995, 14, 133-141; Paulsson et al., Journal of Biological Chemistry 1991, 266, 17545-17551; Manthorpe et al., Journal of Cell Biology 1983, 97, 1882-1890; Schwab et al., Annual Review of Neuroscience 1993, 16, 565-595. Laminin has cell adhesion properties and has been detected in several regions of the embryo including the spinal cord (Azzi et al., Matrix, 9, pp. 479-85 (1989), spinal roots (Rogers et al., Dev. Biol., 113, pp. 429-35 (1986)), optic nerve (McLoon et al., J. Neurosci., 8, pp. 1981-90 (1988)), cerebral cortex (Liesi, EMBO, 4, pp. 1163-70 (1985); Zhou, Dev. Brain Res., 55, pp. 191-201 (1990)), hippocampus (Gordon-Weeks et al., J. Neurocytol., 18, pp. 451-63 (1989)) and the medial longitudinal fasciculus of the midbrain (Letourneau et al., Development, 105, pp. 505-19 (1989)). Accordingly, laminin or peptides derived from laminin can be attached or adsorbed onto the devices of the invention to promote neural cell adhesion and directional growth.

A small domain of an adhesion protein can also be used, for example, the peptide

Arg-Gly-Asp (also referred to a “RGD”) that is found in many adhesion proteins. The RGD peptide is responsible for some of the cell adhesion properties of fibronectin (Pierschbacher and Ruoslahti, Science, 309, pp. 30-33 (1984)), laminin (Grant et al., Cell, 58, pp. 933-43 (1989)), entactin (Durkin et al., J. Cell. Biol., 107, pp. 2329-40 (1988)), vitronectin (Suzuki et al., EMBO, 4, pp. 2519-24 (1985)), collagen I (Dedhar et al., J. Cell. Biol., 107, pp. 2749-56 (1987)), collagen IV (Aumailley et al., Exp. Cell Res., 187, pp. 463-74 (1989)), thrombospondin (Lawler et al., J. Cell. Biol., 107, pp. 2351-61 (1988)) and tenascin (Friedlander et al., J. Cell. Biol., 107, pp. 2329-40 (1988)).

Varying the sequence or flanking sequences of such adhesion peptides can alter the binding affinity of a receptor for the peptide or protein containing it. The density of the biologically active molecule in the pre-selected pattern may affect adhesion, binding and cellular responses, and it will be appreciated that it may be necessary to control the density of the biologically active molecule to obtain the optimum density for practicing the present methods.

Further examples of biologically active molecules contemplated by the present invention include the peptide Tyr-Ile-Gly-Ser-Arg (SEQ ID NO:1), found in the B1 chain of laminin and that binds to the 67 kDa laminin receptor found on many cell types. Peptides having SEQ ID NO:1 promote epithelial cell attachment (Graf et al., Biochemistry, 26, pp. 6896-900 (1987)) and inhibit tumor metastasis (Iwamoto et al., Science, 238, pp. 1132-34 (1987)).

The peptide Ile-Lys-Val-Ala-Val (SEQ ID NO:2) found in the A chain of laminin is also contemplated as a biologically active molecules for use in the invention. Peptides having SEQ ID NO:2 have been reported to promote neurite outgrowth (Tashiro et al., J. Biol. Chem., 264, pp. 16174-182 (1989); Jucker et al., J. Neurosci. Res. 28, pp. 507-17 (1991)). Many different peptides with SEQ ID NO:2 sequence may stimulate neurite extension, however, the isolated SEQ ID NO:2 peptide may not be sufficiently water soluble for all of the present applications. As an alternative, the water-soluble peptide Cys-Ser-Arg-Ala-Arg-Lys-Gln-Ala-Ala-Ser-Ile-Lys-Val-Ala-Val-Ser-Ala-Asp-Arg (SEQ ID NO:3) may be used.

The peptide Arg-Glu-Asp-Val (SEQ ID NO:4) from fibronectin binds to the integrin on human endothelial cells, but does not support adhesion or spreading of smooth muscle cells, fibroblasts or platelets and may therefore be useful for achieving selective cell adhesion.

Cell binding domain sequences of extracellular matrix proteins may also be used as biologically active molecules within the present invention. Examples of such domain sequences include: the Arg-Gly-Asp-Ser (SEQ ID NO:5) peptide sequence found in fibronectin which can mediate adhesion of most cells via the ap receptor; the Leu-Asp-Val and Arg-Glu-Asp-Val (SEQ ID NO:6) peptide sequences from fibronectin which can also mediate adhesion of cells; the Arg-Gly-Asp-Val (SEQ ID NO:7) peptide sequence from vitronectin which can mediate adhesion of most cell types via the ccp receptor; the Leu-Arg-Gly-Asp-Asn (SEQ ID NO:8) peptide sequence from Laminin A that can mediate cell adhesion; the Pro-Asp-Ser-Gly-Arg (SEQ ID NO:9) peptide from Laminin B1 that can mediate cell adhesion; the Arg-Asn-Ile-Ala-Glu-Ile-Ile-Lys (SEQ ID NO:13) peptide from Laminin B2 that can mediate neurite extension; the short Asp-Ala dipeptidyl sequence; the Arg-Gly-Asp-Thr (SEQ ID NO:10) peptide from Collagen I that can mediate adhesion of most cells; the Asp-Gly-Glu-Ala (SEQ ID NO:11) sequence that can mediate adhesion of platelets and other cells; and the Val-Thr-Xaa-Gly (SEQ ID NO:12) of thrombospondin that can mediate adhesion of platelets.

Further examples of biologically active molecules useful in the present invention include epidermal growth factor, nerve growth factor, insulin-like growth factor, basic fibroblast growth factor, platelet derived growth factor, transforming growth factor and related growth factors. Other examples include bone morphogenetic proteins, cytokines including interferons, interleukins, and monocyte chemotactic protein-1. It will be appreciated that the biologically active molecules of the present invention may also be provided on biocompatible, biodegradable polymeric substrates and so that they may be released as the material degrades.

Further examples of biologically active molecules contemplated by the present invention include dopamine, amine-rich oligopeptides, such as heparin binding domains found in adhesion proteins such as fibronectin and laminin. Other examples include amines, basic amino acids, and monosaccharides that can bind to the asialoglycoprotein receptor on hepatocytes. For example, one can stably adsorb N-acetylglucosamine or lactose or a polymerized N-acetyllactosamine monomer to the polymeric substrates of the present invention. Another example is sialyl Lewis X saccharide (Varki, Proc. Natl. Acad. Sci., (USA) 91:7390 (1994)) that is a biologically active molecule for the selectin class of saccharide-binding receptors that are usually responsible for mediating cell-cell interactions (Lasky, Science, 258:964 (1992)). Thus, this saccharide may be useful for mimicking cell-cell recognition.

The suitability of a biologically active molecule for use in the present invention may be assessed by methods known to those skilled in the art. For example, when it is desired to bind a specific biomolecule or cell to an device of the present invention, a potential biologically active molecule is stably adsorbed to a polymeric substrate and the binding or adhesion of a biomolecule or cell can be assessed by observing whether the cell or biomolecule binds to a polymeric substrate, by measuring protein-protein interactions between the biomolecule or cell and the biologically active molecules, or by detecting whether an antibody reactive with the biomolecule or cell becomes bound to the polymeric substrate. Functional assays for detecting whether a cell responds to a biologically active molecule may also be used, for example, functional assays capable of assessing whether the cell grows or adheres to the biologically active molecule under consideration. All of these procedures are available and can be adapted by one of skill in the art to identify biologically active molecules that are suitable for use in the present devices and methods.

Methods for Preparing Devices with Patterns of Biologically Active Molecules

Any method for covalently attaching or stably adsorbing a biologically active molecule onto a substrate can be employed to prepare a therapeutic device of the invention.

In one embodiment, the biologically active molecules are stably adsorbed through non-covalent interactions. Methods for stable adsorption involve treating the surface of a polymeric substrate to increase the ability of the surface to stably adsorb the biologically active molecules. A mixture of biologically active molecules is then transferred to the surface of the substrate where the molecules are directly and stably adsorbed to the polymeric substrate through a polar interaction between the surface and the biologically active molecule.

The surface of the substrate can be treated prior to contact with the biologically active molecule to increase the polarity of the surface. In order to accomplish this, the polymeric substrate is exposed to conditions that temporarily increase polarity of the surface of the polymeric substrate. Any method known to one of skill in the art can be used to increase the polarity of the surface of the polymeric substrate. In preferred embodiments, the polymeric substrate may be made polar by modifying the surface energy or surface tension of organic groups within the polymeric substrate or by temporarily aligning the dipole moments or polarity of the polymeric compounds within the substrate. Polymers having polar groups such as amines, amides, carbonyls, carboxylates, esters, alcohols, sulfhydryls and the like may be preferred substrates for incorporation into the devices of the present invention.

In one embodiment, the polymeric substrate is placed in a low temperature plasma generator and exposed to an ionized gas plasma for a time and at a temperature and pressure that temporarily increase the polarity of surface of the polymeric substrate. For example, the polymeric substrate can be exposed to a stream of plasma for a temperature and under an electrical wattage sufficient to make the surface of the polymeric substrate polar. The plasma pressure can vary depending on the type of plasma, the temperature and other factors. For example, a pressure of about 10 to about 500 ton, preferably about 100 to about 300 torr and more preferably about 150 to about 250 ton can be used One of skill in the art can also vary the time of such exposure as needed to make the surface of the polymeric substrate polar. For example, the time of exposure to plasma can include any suitable time, such as for example from about 0.5 to 300 seconds, preferably from about 1 to 200 seconds and more preferably from about 5-120 seconds. One of skill in the art can also readily determine a temperature sufficient to make the surface of the polymeric substrate polar. For example, convenient temperatures for making the polymeric substrate polar are about 5° C. to about 42° C., preferably about 10° C. to about 37° C. and more preferably about room temperature. An electrical wattage sufficient to make the surface of the polymeric substrate polar varies with the type of gaseous plasma. For example, such wattage can vary from about 5 to 500 Watts, preferably about 50-400 Watts and more preferably about 100-300 Watts. Convenient conditions for making the surface of the polymeric substrate polar include exposing the device to oxygen plasma for about 60 seconds, at 160 torr oxygen, and using 200 Watts and at room temperature.

Any ionic gaseous plasma known by one of skill in the art to activate the surface of the polymeric substrate can be used. Types of plasmas contemplated by the present invention include argon, nitrogen, oxygen, and other gases known to those of skill in the art to readily be ionized. Preferably the surface is treated with oxygen plasma.

Polymeric substrates treated in the manner describe were evaluated by cell attachment assays, cell alignment assays, x-ray photoelectron spectroscopy, atomic force microscopy, scanning electron microscopy and near-field scanning optical microscopy. When using the present methods to make the surface of the polymeric substrate polar, it was observed that the surface was not significantly chemically changed.

However, while such treatment does not substantially alter the chemical composition of the polymeric substrate, it does transiently make the polymeric substrate sufficiently polar in nature for adsorption of a defined, pre-selected pattern of biologically active molecules. Moreover, such treatment does not make the polymeric substrate so hydrophilic or so polar that the applied pattern of biologically active molecules becomes obscured by diffusion. Such treatment also transiently activates or polarizes the surface of the polymeric substrate for a sufficient time to apply a pre-selected pattern of biologically active molecules, for example, for about two weeks, after which time the substrate will return to its original state.

Transferring a pre-selected pattern of biologically active molecules to the surface of a polymeric substrate generally involves contacting that surface with one or more biologically active molecules such that the biologically active molecules are retained on the surface of the substrate in a pre-selected pattern through a non-specific molecular interaction.

Any method used by one of skill in the art to apply a pattern of biologically active molecules to a polymeric substrate can be used. For example, biologically active molecules can be applied using a roller, printer, stamp or similar apparatus. According to the present invention, all of these methods are termed “stamping” the biologically active molecules onto the polymeric substrate. Stamping methods of the present invention allow for production of multiple micropatterned polymeric substrates in minimal amounts of time (e.g. minutes). In contrast, preparation of multiple micropatterned polymeric substrates via prior art microlithography can take several hours to days of work.

In order to transfer a pattern of biologically active molecules to a polymeric substrate, the stamp is first coated with biologically active molecules. However, only biologically active molecules in the stamp pattern are transferred to the substrate. Preferably the stamp pattern is a raised pattern, but any method known to one of skill in the art for making a pattern on a stamp is contemplated. Preferably the pattern is micron-sized. Upon pressing the stamp to the surface of the polymeric substrate, the pattern of biologically active molecules is transferred to the polymeric substrate. Hence, the biologically active molecules are stamped onto the surface of the substrate in a spatially controlled manner. The micron-sized pattern can be any pattern contemplated by one of skill in the art, including a line, circle, oval, square, rectangle, diamond, triangle or a combination of these shapes. Selection of a pattern is dependent upon the application that the device is intended to be used. For example, in one embodiment the pattern is a line. Biomolecules and cells may adhere to the biologically active molecules in the shape of a line so that a linear pattern of cells can be formed or so that cells placed at one end of the line will grow toward the other end of the line. The line can have any width of use to one of skill in the art and can vary with the type of tissue or the extent of tissue damage.

For example, for micron-sized patterns, a line can be about 1 to about 10,000 microns in width, or about 100 to 1000 microns in length. In some embodiments, such lines can be about 1 to about 80 microns in width and about 500 to about 1500 microns in length. More preferred line patterns are about 30 microns to about 1000 microns in size.

As described above, a stamp may be used to contact the surface of the polymeric substrate with the biologically active molecules. The stamp is dipped in an aqueous solution of the biologically active molecules. A preferred vehicle for the biologically active molecules is phosphate buffered saline solution or the like. The stamp is then placed in contact with the surface of the polymeric substrate to be patterned and left for approximately 1-120 minutes, preferably about 15 minutes, so that the biologically active molecule transfers from the stamp to the substrate. The stamp is then removed and the patterned substrate can be used immediately or stored for future use.

A stamp use for in the present invention can be made by any method known to one of skill in the art. One procedure for making a stamp involves the preparation of a master that has the reverse image of the micron-sized pattern to be placed on the stamp.

Briefly, a master may be fabricated on a polished silicon wafer by pouring SU-8 photoresist (Microchem Corp.) to a thickness of about 25 μm and the master is processed by contact photolithography. Methods for the production of masters are known in the art. See, Moread, W.M., Semiconductor Lithography: Principles and Materials, Plenum, New York (1988); Brambley et al., Adv. Mater. Opt. Electron., 4:55 (1994); Handbook of Microlithography, Micromachining, and Microfabrication, Vol. 1 (Ed: P. Rai-Choudbury), SPEI Optical Engineering Press, Bellingham, Wash. (1997).

Any material known to one of skill in the art can be used to make the stamp. A preferred material for the stamp is poly(dimethyl siloxane) (PDMS). One source of PDMS material is Sylgard 184™ (Dow Corning, Midland, Mich.). The flexibility of a stamp prepared from this material permits patterning of non-flat surfaces such as round or cylindrical polymeric substrates. For PDMS stamp preparation, the PDMS material is first prepared in liquid form and mixed with the curing agent. The mixture is poured over a master pattern. It is preferred that the master pattern comprises an organic or inorganic material such as glass, which remains hard at temperatures greater than 60° C. Any bubbles are removed preferably via a vacuum (e.g. at about 28″ Hg). This mixture is then permitted to cure at approximately 60° C. for a minimum of 4 hours. The resulting stamp is released from the master pattern upon curing. The stamp is treated with a plasma as described above to make it more hydrophilic or polar so that the selected biologically active molecules will adsorb to it.

When the entire interior surface of a therapeutic device is to be coated with biologically active molecules a stamp may not be needed. Instead, the interior surface of the therapeutic device may be treated as described above to make the surface transiently hydrophilic A solution of biologically active molecules can then be applied or flushed through the therapeutic device so that the biologically active molecules can adsorb to the surface.

One of skill in the art can readily vary the foregoing procedures to generate therapeutic devices with stably adsorbed or covalently attached biologically active molecules.

Methods for Spatially Direct Cellular Growth with the Present Devices.

Currently available degradable or absorbable devices for tissue regeneration can cause local inflammation. However, polymeric substrates of anti-inflammatory agents actually decrease local inflammation and/or pain. According to the invention, these polymeric substrates can be incorporated into therapeutic devices to provide preformed or adaptable scaffolding for tissue repair and regeneration, including repair of neural, muscle, ligament, tendon, bone and other tissue damage. Further, it has been found that these devices promote the healing process of tissues through enhanced regeneration of these tissues.

The invention therefore also provides methods for using the present devices for spatially directing tissue or cell growth, including for neurite outgrowth and neural tissue regeneration. The present methods allow tissue regeneration and outgrowth of neural cells through adhesion and/or growth stimulation along the specific pattern of biologically active molecules on the surface of the devices. Polymeric devices having biologically active molecules adsorbed or attached are particularly useful tissue engineering applications because the present devices not only act as templates to guide and regulate cell growth after promoting cell adhesion but they slowly release beneficial anti-inflammatory agents to promote healing and prevent inflammation.

A device of the present invention is thus employed as a template for tissue regeneration and also as controlled release device for sustained delivery of anti-inflammatory agents to a desired site. The quantity of anti-inflammatory agent that hydrolyzes from the polymeric substrate of the device can be readily determined by those of ordinary skill in the art. The quantity selected for use in the device relates to the amount needed to produce an effective treatment over a given time period. The device can readily be tested to ascertain how much anti-inflammatory agent is released over time. The size and weight of the device is then adjusted to provide an amount corresponding to the amount of anti-inflammatory agent needed over a known time period.

The present invention provides methods for spatially modulating the growth of a tissue, a cell or a nerve cell process that includes contacting a tissue, or cell with a device of the present invention for a time and under conditions sufficient to adhere the tissue or cell to the biologically active molecules on the device and to permit growth of the tissue, cell or nerve cell process along a pattern of biologically active molecules on the polymeric substrate of the device.

The devices of the invention may be used in any mammal. However, it is preferred that the mammal is a human. In some embodiments, the device is used for repair and regeneration of nervous tissues. In order to regenerate tissue into a desired shape, mammalian tissues are encouraged to grow along the surface of adsorbed or covalently bound biologically active molecules.

The present methods involve contacting the surface of the device of the invention with a cell such that the cell adheres to a biologically active molecule provided on the surface of the device. Conditions sufficient to adhere the cell to the biologically active molecules on the device and to grow the cell along the pattern of biologically active molecules on the polymeric substrate of the device include cell culture conditions and conditions permitting growth of selected cell types. The cell can be contacted with the device in vitro or in vivo. Preferably the cell is contacted with the device in vivo by implanting the device into a specific site. The site chosen can be any site where repair of tissue is needed, or where tissues or nerves need to be regenerated or directionally guided in their growth.

According to the present invention, molecular interactions between neurons and the pattern of biologically active molecules of the present devices encourages neurite extension. Devices prepared according to the invention with particular cell adhesion proteins, growth factors and other biologically active molecules are beneficial for promoting such neurite extension. Such devices preferably are patterned as a hollow tube, jellyroll or rod of polymer with biologically active molecules that promote neurite extension adsorbed inside or outside the tube.

Tissue regeneration or engineering can be initiated outside the body by, for example, removing cells from a patient and seeding those cells onto the device in an appropriate culture medium. When the cells have grown, divided and/or differentiated to form a tissue in culture, the new tissue may be implanted into the body. The device may be implanted at any stage in the growth of the tissue, depending on clinical need. However, because the anti-inflammatory polymeric substrates promote healing and tissue regeneration, surgical implantation can be performed soon after seeding of cells into the device.

For nerve regeneration applications, patterns composed of the biologically active molecules that include peptide sequence Ile-Lys-Val-Ala-Val (SEQ ID NO:2) may be used to encourage nerve cell growth to follow predetermined pathways, i.e. between two severed points of a nerve or towards a de-nerved tissue. Such biologically active molecules can be covalently bound or adsorbed onto selected portions or the entire interior surface of a therapeutic device of the invention. Alternatively, other patterns of such biologically active molecules can be provided on the therapeutic devices of the invention, for example, continuous stripes along the length of the therapeutic device.

A method of forming a device for regenerating tissue according to the present invention may be carried out substantially as described below. First, a biocompatible, biodegradable polymeric substrate is chosen that degrades into anti-inflammatory agent over a desired time period. Second, a type of biologically active molecule or a mixture of biologically active molecules is selected that will provide the desired functions, for example, cell adhesion and/or cell growth. Third, a spatially controlled pattern of the selected biologically active molecules is stamped on or applied to an activated surface of the polymeric substrate. In order to regenerate a tissue, the device can be surgically implanted at the appropriate site or cultured in vitro with surgically removed cells and then surgically implanted, as described above.

An advantage of the present devices is that the non-coated surface of the substrate can be hydrophobic. This property decreases the ability of cells and other biomolecules to adhere to the non-covered regions of the polymer substrate. Hence, cells adhere more specifically to the regions of the polymeric coated by biologically active molecules and the device is therefore better able to direct cell growth. Another advantage of the invention is that cells may be grown in vitro under common laboratory conditions or cells may be grown in vivo upon implantation of the device into a living mammal.

Devices comprising the patterned polymeric surfaces prepared via the method of the present invention can direct outgrowth of neurons for the repair of peripheral and central nervous system damage. Hence, peripheral nerves, spinal cord tissues and other nerves can be treated using the methods and devices of the invention. When injury occurs to the nerve, regrowth of the affected axons must be directed along their original path for function to resume. In the body, both physical and chemical cues direct regrowth. Substrates prepared in accordance with the process of the present invention can mimic some of these cues, thereby encouraging nerve cell alignment and regrowth.

The following non-limiting examples are provided to further illustrate certain aspects of the invention.

### Example 1

### Optimized Synthesis of Salicylate-Based Poly(Anhydride) Esters

The synthesis of a salicylate-based poly(anhydride-ester) was optimized to improve the overall efficiency of the synthetic process as well as the quality of the polymer. The new approach for the preparation of the polymer precursor minimized the overall number of synthetic steps and increased the overall yield. A new melt-polymerization apparatus was used to provide dynamic mixing, which yielded polymer with increased molecular weights on both the milligram and gram scale.

### Materials

Proton nuclear magnetic resonance (¹H-NMR) spectra were recorded on either a Varian 200 MHz or 300 MHz spectrometer. Samples (5-10 mg) were dissolved in the appropriate deuterated solvent (CDCl₃ or DMSO-d₆) using the solvent as the internal reference. Infrared (IR) spectra were measured on a Mattson Series spectrophotometer by solvent-casting samples onto a NaCl plate. Melting points (Tₘ) were determined on a Thomas-Hoover apparatus.

### Methods

Weight-averaged molecular weights (M_{w}) and polydispersity indices (PDI) were determined by gel permeation chromatography (GPC) on a Perkin-Elmer (PE) LC system having a Series 200 refractive index detector, a Series 200 pump, and an ISS 200 autosampler. A DEC Celebris 466 computer running PE TurboChrom 4 software was used for data collection and processing, and to automate the analysis via PE-Nelson 900 Interface and 600 Link. Samples (5 mg/ml) were dissolved in THF, filtered through 0.45 μm poly(tetrafluoroethylene) (PTFE) syringe filters (Whatman, Clifton, N.J.) and immediately injected. Samples were resolved on a Jordi DVB mixed-bed GPC column (7.8×300 mm) (Alltech, Deerfield, Ill.). Molecular weights were calibrated relative to narrow molecular weight polystyrene standards (Polysciences, Dorval, Canada).

The reaction scheme employed is depicted below.

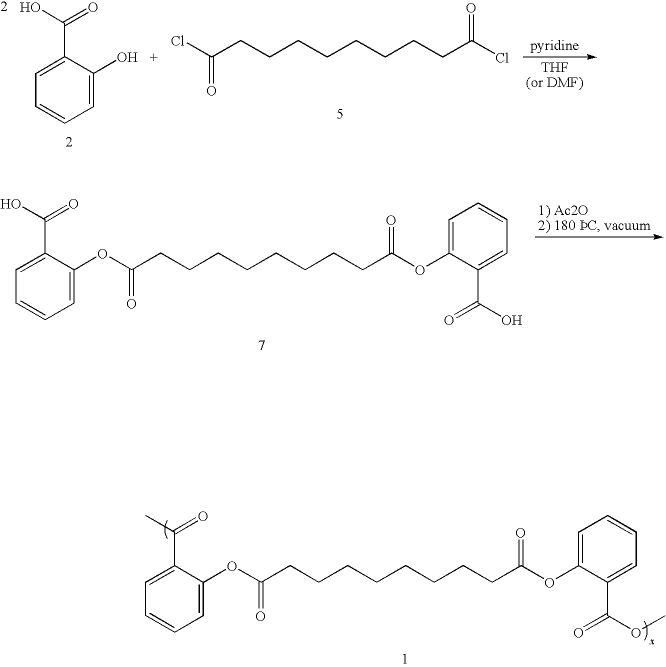

Salicylic acid (2; 1.2 g, 8.4 mmol) was dissolved in a solution of THF (3.0 ml) and pyridine (9.0 ml). Sebacoyl chloride (5; 1.0 g, 4.2 mmol) was added dropwise via syringe over 5 minutes to the stirring reaction solution set in an ice bath (˜0° C.). The reaction mixture was warmed to room temperature, stirred for 2 more hours, then poured over an ice-water slush (150 ml). After acidifying to pH˜2 with concentrated HCl, compound 7 was isolated by vacuum filtration, washed with water (3×50 ml), and air-dried. Yield: 97% (white powder). ¹H-NMR (CDCl₃): δ 8.13 (d, 2H, ArH), 7.61 (t, 2H, ArH), 7.35 (t, 2H, ArH), 7.12 (d, 2H, ArH), 2.63 (t, 4H, CH₂), 1.82 (m, 4H, CH₂), 1.48 (b, 8H, CH₂). IR (NaCl, cm⁻¹): 3400-2700 (COOH), 1760 (C═O, ester), 1700 (C═O, ester). Anal. Calcd: C, 65.18; H, 5.88. Found: C, 64.50; H, 5.73. Tₘ=128-131° C.

The diacid (7) was activated into monomer prior to polymerization using previously described methods. Campo C, Anastasiou T, Uhrich K (1999) Polym Bull 42:61; Anastasiou T, Uhrich K (2000) Macromolecules 33:6217. In brief, the diacid (7) was added to an excess of acetic anhydride (100 ml), then stirred at room temperature until a homogenous solution was observed (approximately 120 min). The monomer is isolated by removing excess acetic anhydride under vacuum, and then washed with diethyl ether (50 ml). Monomer (500 mg) was placed in the appropriate reaction vessel, which was heated to 180° C. using a temperature controller (Cole Parmer) in a silicone oil bath under high vacuum (<2 mmHg) for 1 to 3 h. During this time, the melt was actively stirred at ˜100 rpm by the overhead stirrer (T-line Laboratory Stirrer, Talboys Engineering). Polymerization was complete when the viscosity of the melt remained constant and/or solidified. The polymer was cooled to room temperature, dissolved in a minimal volume of methylene chloride (15 ml), and precipitated into a 20-fold excess of diethyl ether (300 ml). Yield: quant. (pale tan solid). ¹H-NMR (DMSO-d₆): δ 8.20 (d, 2H, ArH), 7.95 (t, 2H, ArH), 7.75 (t, 2H, ArH), 7.40 (d, 2H, ArH), 2.20 (t, 4H, CH₂), 1.55 (m, 4H, CH₂), 1.25 (b, 8H, CH₂). IR (NaCl, cm⁻¹): 1792, 1740 (C═O, anhydride), 1760 (C═O, ester).

### Results and Discussion

The method previously developed by the inventors to synthesize the diacid (7) (which is the monomer precursor to polymer 1), yielded moderate quantities of product. Erdmann L, Uhrich K. (2000) Biomaterials 20:1941. Synthetic methods that would provide diacid (7) in fewer reaction steps, with minimal purification, and higher overall yields were explored.

By modifying a method reported by Pinther and Hartmann, the optimal reaction conditions were defined: the free salicylate (2) was directly coupled with the diacyl chloride (5) in an appropriate solvent containing pyridine to give the diacid 7. See Pinther P, Hartmann M (1990) Makromol Chem Rapid Commun 11:403. Pinter and Hartmann converted aliphatic acids into acyl chlorides by treatment with thionyl chloride, and the diacyl chlorides were subsequently reacted with 4-hydroxybenzoic acid in the presence of stoichiometric amounts of pyridine in dioxane.

Because the polymers of the invention may be used for medical purposes, the solvent dioxane was not used and the pyridine concentration was decreased. Although several solvents were evaluated, tetrahydrofuran (THF) was selected as a low-boiling, polar solvent.

A stoichiometric amount of pyridine was used to deprotonate the salicylate (7). The pyridine also acts as a catalyst to form an acyl pyridinium ion (Ferscht A, Jencks W (1970) J Amer Chem Soc 92:5432), which reacts with the free phenolate. Therefore, formation of the acyl pyridinium ion, which is known to react more rapidly with alcohols than acyl chlorides, eliminated the need to protect the carboxylic acid of the salicylate (2). This method also eliminated further purification steps, except for washing with an appropriate solvent, because of the large solubility differences between the product (7) and the reaction's potential by-products. Resultant conversions were quantitative and isolated yields were greater than 90%.

This one-step procedure has been applied to a variety of related diacids, where aminosalicylates are used in place of salicylic acid (2) and various alkyl- and aryl-based acyl chlorides have been used in place of the sebacoyl chloride (4). Given the simplicity and ease of diacid isolation, this one-step method is a preferred choice for preparing a variety of diacids that will undergo melt condensation to yield poly(anhydride-esters).

### Polymer Synthesis

The polymerization apparatus was redesigned to give higher molecular weight materials on both the milligram and gram scale. In initial synthetic procedures, the polymerization methods employed were similar to those described by Domb A, Langer R (1987) J Polym Sci, Part A: Polym Chem 25:3373; and Domb A (1992) Macromolecules 25:12. In brief, the poly(anhydride-esters) (1) were synthesized by melt condensation polymerization using a side-arm test tube containing a magnetic stir bar, attached to a gas-vacuum manifold. The monomers were melt-polymerized at 180° C. under vacuum (<2 mmHg) until the melt solidified, and the reaction vessel was flushed every 15 min with dry nitrogen with stirring. Incomplete mixing, due to increased viscosity of the polymer melt after the reaction proceeded, resulted in prolonged polymerization times and low molecular weights, even at the milligram scale. In addition, portions of the polymer melt would locally decomposed due to incomplete mixing, resulting in polymers that were dark brown and sometimes charred.

To overcome these issues, other methods were investigated to provide more homogenous mixing of the melt. Simple, inexpensive, and readily available components were selected to make a polymerization apparatus that is described in Schmeltzer et al., Polymer Bulletin 49: 441-48 (2003). This apparatus actively stirred the molten monomer, while maintaining a high vacuum (<2 mmHg). Both small (<1 g) and medium (1 g-100 g) scale models used a typical laboratory stirring motor.

On the small scale (<1 g), the polymerization vessel was constructed from microscale glassware components with 14/10 joints (see Schmeltzer et al., Polymer Bulletin 49: 441-48 (2003)). A cylindrical bottom vial (10 ml) was equipped with a vacuum adapter; the included o-rings and screw-top joints ensured a vacuum seal, and created a modular system. The stirring shaft was constructed by shaving the edges of the spoon end of a stainless steel lab spoon-spatula (9″) to fit through the 14/10 joint of the vial. The spatula end was flat, which allowed the shaft to interlock with the stirring motor. The joint and o-ring at the top of the vacuum adapter formed a vacuum-tight fit around the shaft. Sealing the vessel with a septum facilitated storage of the final polymer.

On the medium scale (1 g-100 g), the polymerization apparatus was similar but 125-250 ml two-necked round-bottom flasks with 24/40 joints were used as the reaction vessel (see Schmeltzer et al., Polymer Bulletin 49: 441-48 (2003)). A vacuum joint was installed in one neck, while the other neck held a Teflon vacuum-stirring adapter. The stirrer assembly consisted of a glass stirring shaft and Teflon paddle (19 mm×48 mm) After the polymerization, a standard-taper stopper sealed the flask.

Several changes were observed when these changes in the polymerization technique were employed. First, using the modular apparatus in conjunction with the overhead mechanical stirrer, poly(anhydride-esters) were prepared with weight-averaged molecular weights around 30,000. In contrast, using the earlier system (i.e., magnetic stirrer), polymer molecular weights were typically below 10,000. Second, slightly elevated glass transition temperatures were observed with the increased molecular weights. For poly(anhydride-ester) described herein that were comprised of salicylate and sebacate, the glass transition temperature was raised from 23.5° C. (magnetic stirrer) to 27.0° C. (mechanical stirrer). This aspect was extremely important for the continued development of homopolymer 1, because glass transition temperatures above room temperature significantly enhance the processing capabilities of these polymeric materials. Third, the poly(anhydride-esters) were consistently white in color, as opposed to brown, which is an important indicator of purity and is especially significant for using homopolymer 1 in medical applications.

### Example 2

### In Vivo Implantation of Anti-Inflammatory Polymeric Substrates Promotes Healing

In vivo studies were conducted to compare the affects of a polymeric substrate of the present invention (referred to herein as the bioactive polymer or implant) and a chemically similar polyanhydride (referred to herein as the control polyanhydride) on the healing process. The sole chemical difference between these two polymers was the replacement of the ether bond in the polyanhydride of the bioactive polymer with an ester bond. This difference results in degradation to salicylic acid by the bioactive polymer as compared to degradation to a non-active component in the control polyanhydride.

In these experiments, the polymers were compression-molded into films with thicknesses of 0.1, 0.2 and 0.3 mm and cut into 0.5 mm wide strips.

Mice (n=10) were anesthetized and the palatal gingival mucosa adjacent to the maxillary first molar was reflected to expose the palatal and alveolar bone. A polymer film was then placed on the bone adjacent to the tooth. The tissue was repositioned and the procedure was repeated on the contra lateral side. Polymer films were randomly placed (left vs. right) with each mouse carrying both polymers. Mice were fed a ground diet and water ad libitum and weighed weekly. Mice were sacrificed at 1, 4 and 20 days post-surgical insertion.

Visual intraoral examination of the mucosa covering the implantation sites was performed with a dissecting microscope under optimum lighting. Magnification was varied from 5 to 40 times normal size. Photographs were taken to record the morphological changes observed.

Polymer membranes of thicknesses 0.1 and 0.2 mm were not visible under the microscope at 4 and 20 days post insertion. However, thicker membranes (0.3 mm) were still observable after 20 days.

In mice receiving the control polyanhydride films, the mucosa was red and thin near the implant with the surrounding tissue inflamed at days 1 and 4. By day 14, the tissue was slightly puffy in three animals and within normal limits for the remaining 5 animals. In contrast, the tissue surrounding the bioactive polymer implants was slight puffy after day 1 but within normal limits in all animals by day 4.

In general, considerable swelling was observed on the side bearing the control polyanhydride, whereas the side with the bioactive polymer showed a progressively normal mucosa. The tissue surrounding the control polyanhydride was very swollen and white, whereas the tissue adjacent to the bioactive implant was less swollen and normal in color. The three maxillary molar palatal ridges (anterior, middle and posterior) were clearly visible. However, the anterior and middle ridges coalesced because of the swelling and blanching on the control polyanhydride side. This effect was most pronounced at day 13. By days 15 and 20, blanching and swelling on the control polyanhydride side were considerably diminished.

Histological examination of tissues from the mice was also performed. After sacrifice, tissues were fixed in 10% formalin, decalcified, embedded in paraffin, sectioned serially at 4 μm thickness, and stained with hematoxylin and eosin. The sections were subjected to microscopic evaluation and histometric assessment using 4, 10 and 20× magnifications. The histopathological examination correlated well with visual observations.

One mouse was sacrificed 24 hours post implantation. The histology showed heavy infiltration of polymorphonuclear (PMN) leucocytes and erythrocytes. The 0.1 mm films were mostly dissolved during the tissue processing procedure. The bone was denuded from the periosteum and the polymer was in direct contact. The gingival epithelium and connective tissue below the subcular epithelium was broken. The coronal part of the periodontal ligament linking the alveolar bone to the coronal cementum was mostly intact. The method for reflecting the palatal mucosa was effective in not damaging the periodontal ligament below the level of the bone and coronal cementum. There was no significant difference between the bioactive and control side except for the decrease in swelling on the bioactive side.

Two mice were sacrificed four days post implantation. At this time point, some polymeric material remained in all sites. The 0.1 mm film was in direct contact with the palatal bone. An extensive, thin layer of palatal epithelium was observed that surrounded portions of the polymer specimens. The extent of the epithelium along the membranes was greater for the bioactive than for the control polyanhydride site. Similarly, the PMN cells inflammatory infiltrate was greater on the control polyanhydride side than on the bioactive polymer side. The infiltrate was denser below the epithelium adjacent to the membrane. The infiltrate along the palatal bone was much less.

Six mice were sacrificed at twenty days post implantation. At this time point, small remnants of a 0.3 mm film in only one specimen were present; all other specimens were devoid of polymer. Gingival epithelium including subcular and junctional were essentially restored on all sites. Two specimens showed external resorption that involved cementum and dentin on the control polyanhydride side. Tissue specimens with bioactive polymer showed no alveolar bone, cementum and dentin resorption. However, a significant amount of new bone could be observed coronal to the reversal lines in the sites bearing bioactive films. New bone was also found in the control polyanhydride sites, but at insignificant amounts as compared to the bioactive polymer side. Inflammatory cell infiltrate was present and consisted primarily of PMN cells and macrophages. No erythrocytes were observed except within the vasculature. The intensity of the infiltrate was lower on the bioactive polymer sites.

Quantitative analyses were also performed via electronic images taken of the tissue sections using a Kodak MDS-120 camera attached to an Olympus CH-triocular microscope at magnifications of 4×, 10× and 40×. Using NIH Images 1.61 software, the area of bone, connective tissue, epithelium and artifacts at the lowest magnification were determined. Perpendicular to the widest part of the tooth, a square box was drawn with sides 575 pixels in length. The areas of bone, connective tissue, epithelium and artifacts were determined by the number of pixels within the defined box. All images were blindly analyzed. Sections were taken from mice sacrificed after 20 days from membranes that were either 0.3 or 2 mm thick. Results are shown in Table 2.

TABLE 2

| | Bone Area | Bone Area |
|---|---|---|
| Polymer | (0.3 mm) | (0.2 mm) |
| Control Polyanhydride | 94,750 | 85,563 |
| Bioactive Polymer | 129,637 | 99,702 |

These experiments demonstrate that implantation of a film comprising an aromatic polyanhydride that hydrolyzes to form a therapeutically useful anti-inflammatory agent (a salicylate) resulted in less swelling in tissues adjacent to the film and a decrease in the density of inflammatory cells as compared to other polyanhydride films. Further, little or no bone resorption was observed in the regions near the film as indicated by increased thickness of the palatal.

### Example 3

### Stable Adsorption of Biologically Active Molecules

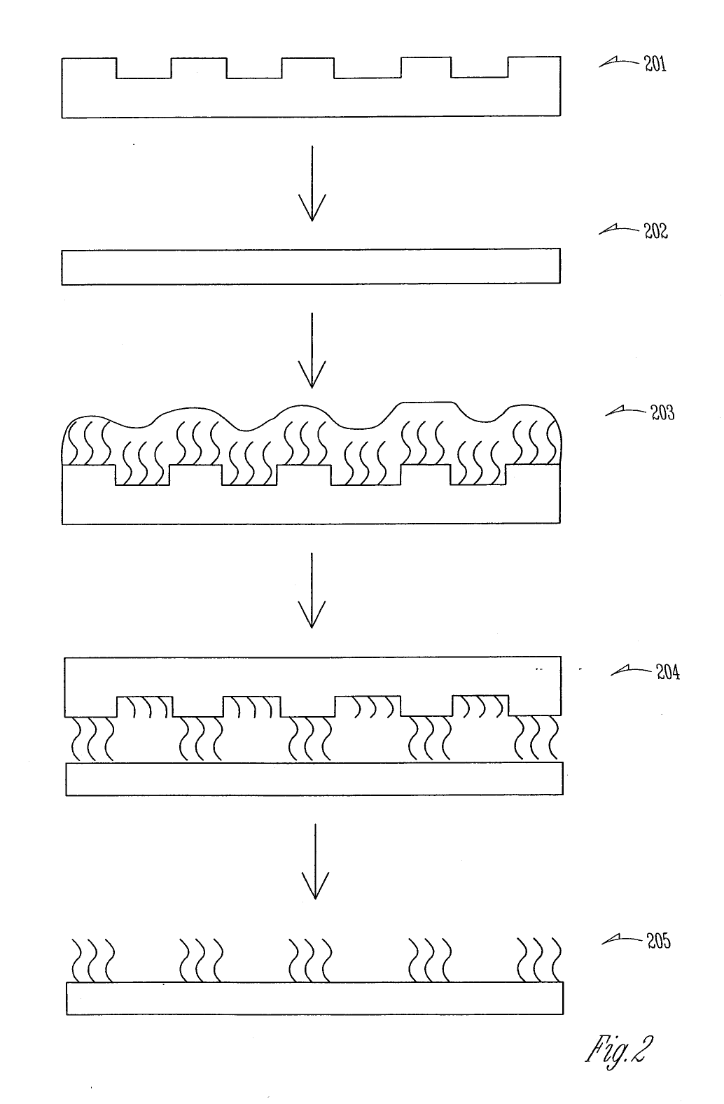

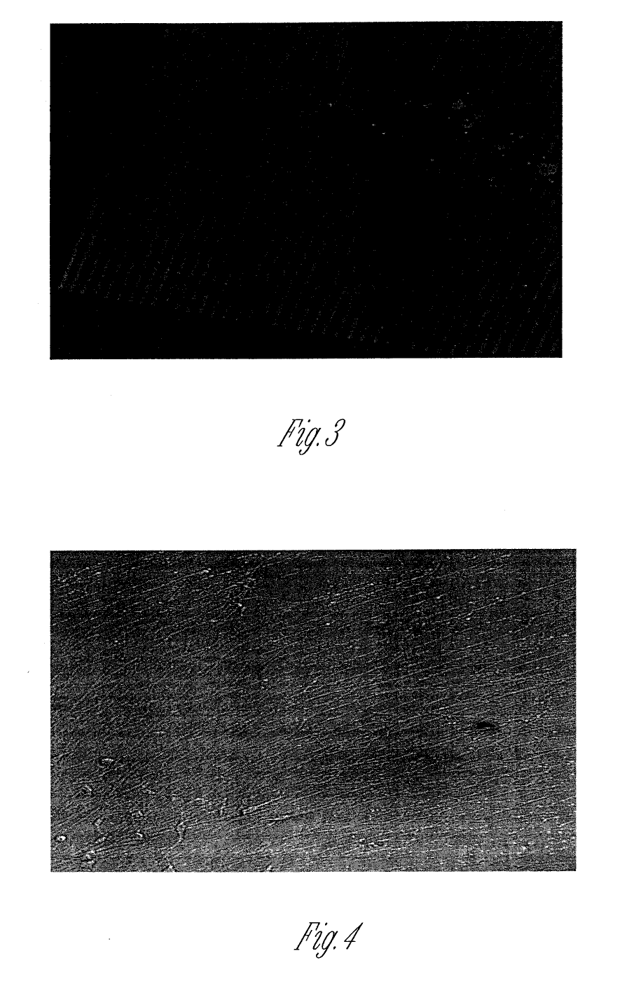

Biologically active molecules were stably adsorbed onto a polymeric substrate without chemical modification of the substrate and without the use of linkers. A nondegradable polymer, poly(methyl methylacrylate) (PMMA, 105 mm thickness, Goodfellows) and a degradable polymer, such as poly(hydroxybutyrate (PHB), polycaprolactone or polycaprolactam), were used as polymeric substrates for application of a micropattern of laminin. See FIG. 2 for the general procedure. For these experiments, the polymers were compressed into thin films of 200 μm and cut to the size of coverslips. After activation, polymeric substrates were evaluated via x-ray photoelectron spectroscopy, and/or scanning electron microscopy. These observation indicated that the polymeric surfaces were not significantly changed. After laminin absorption, its deposition was determined by incubating the prepared slips with rabbit anti-laminin affinity isolated antibody and FITC-conjugated goat anti-rabbit IgG (see FIG. 3).

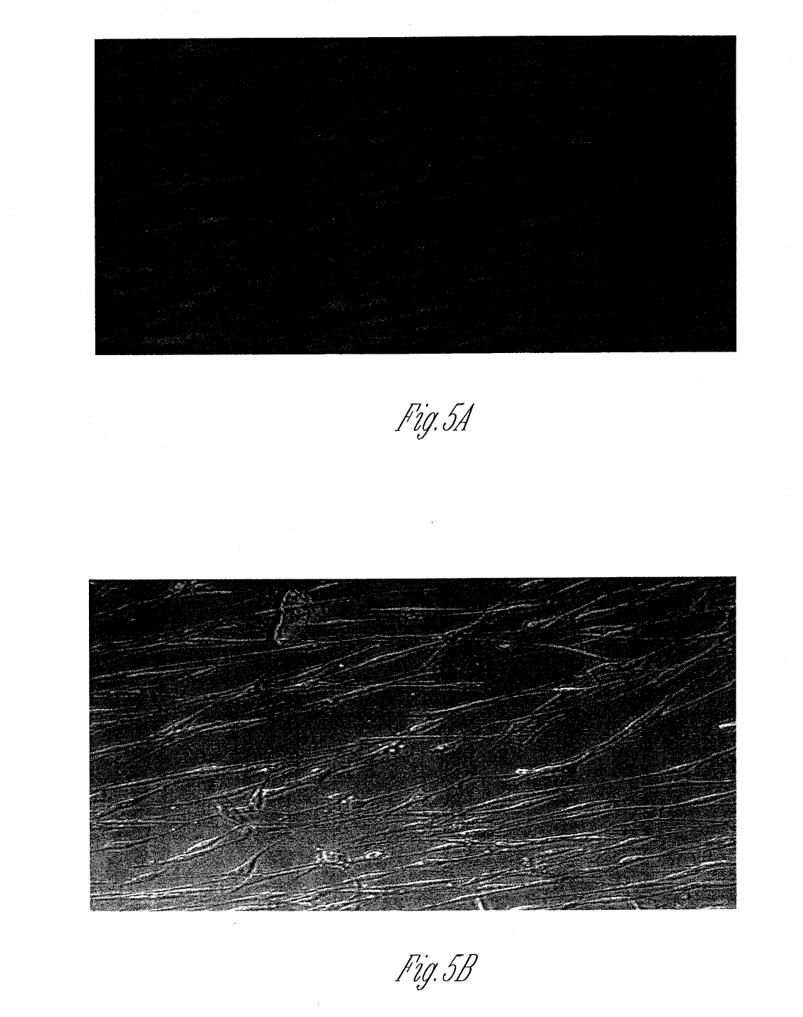

Dorsal root ganglia dissected from seven-day old chick embryos were either directly plated onto the patterned substrate or dissociated into individual neurons and plated. The prepared slips were imaged. Alignment of neural cells on the patterned surface was observed via confocal microscopy (see FIG. 4).

### Master Preparation

Masters used to cast stamps were prepared by methods known in the art. (Moread, W.M., Semiconductor Lithography: Principles and Materials, Plenum, New York, 1988; Brambley et al., Adv. Mater. Opt. Electron., 4:55 (1994); Handbook of Microlithography, micromachining, and Microfabrication, Vol. 1 (Ed: P. Rai-Choudbury), SPEI Optical Engineering Press, Bellingham, Wash. (1997)). Briefly, a master may be fabricated on polished silicon wafers using SU-8 photoresist (Microchem Corp.) at a thickness of about 25 μm. The master is then processed by contact photolithography.

### Polymer Preparation

PMMA was obtained from Goodfellows and used as received. PMMA (100 mg) was compressed into thin films between highly polished steel plates at 10,000 pounds for 95 seconds using a laboratory press (Carver, Wabash, Ind.) heated to 150° C. The press was then cooled to 90° C., after which the plates were immediately removed and further cooled. The polymer film, with a thickness of 150 to 200 mm, was cut into 2-cm squares.

For sterilization prior to cell culturing, the substrates were exposed to UV light at 254 nm (Spectrolinker XL-1500 UV Spectronics, Westbury, N.Y.) at 1200×100 μJ/cm2 for 300 sec.

### Polydimethylsiloxane (PDMS) Stamp Preparation

The master is placed in a petri dish. In a separate container, PDMS monomer (Sylgard 184, Dow Corning, Midland, Mich.) is mixed with the curing agent provided with the PDMS monomer at a 10:1 ratio by weight. Bubbles arising from the mixing process are removed in a vacuum oven (Sheldon MFG, Aloha, Oreg.) at 28″ Hg and at room temperature. This mixture is then poured over the master; any arising bubbles are removed via vacuum at 28″ Hg; and the mixture and master are baked in an oven at 60° C. for a minimum of 4 hours. The resulting PDMS stamp is released from the master by cutting the PDMS with a sharp blade and peeling it from the master.

### Stamping Procedure

The PDMS stamp is placed along with PMMA in a low temperature plasma cleaner (March Plasma). To temporarily increase polarity, the PDMS stamp and PMMA are exposed to oxygen plasma (160 ton) for 60 seconds at 200 W and room temperature. Laminin solution (50 mg/ml, Collaborative Biomedical, Bedford, Mass.) is pipetted directly onto the PDMS stamp or the stamp is dipped in the solution. Any excess solution is then removed via a stream of gas such as nitrogen. The PDMS stamp is then placed in contact with the PMMA and left for approximately 15 minutes to transfer the laminin onto the PMMA. The stamp is then removed. The laminin patterned PMMA can be used immediately or stored in Hank's balanced salt solution (HBSS, Gibco).

### Cell Culture

The laminin stamped PMMA squares were rinsed with Hank's balanced saline solution and plated with dorsal root ganglia or dissociated neurons in minimal, serum-free media. The samples were incubated at 37° C. for 24 to 48 hours, imaged with a Zeiss laser scanning confocal microscope, and the images analyzed for pattern adherence. Cells had adhered to the laminin pattern on the PMMA substrate (FIG. 4). Moreover, neuronal processes also adhered and grew along the laminin pattern on the PMMA substrate (FIG. 4).

Purified primary rat Schwann cells were obtained from Prof. M. B. Bunge, University of Miami, the Miami Project to Cure Paralysis, and maintained in tissue culture flasks with a growth media consisting of Dulbecco's Modified Eagle's Medium (DMEM, Sigma), 10% v/v fetal bovine serum (Gibco), L-glutamine (2 mM, BioWhittaker), penicillin/streptomycin (P/S, 50 U/mL/50 μg/mL, BioWhittaker), 10 μg/mL bovine pituitary extract (Collaborative), and 10 μM forskolin (Sigma).

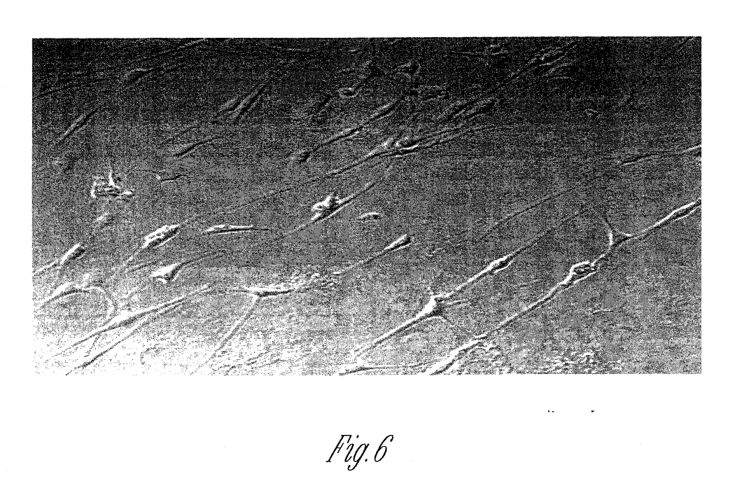

Schwann cells were rinsed with phosphate buffered saline (PBS, BioWhittaker) and incubated with trypsin (2.5 g/mL, Sigma) for 3 min. An equal amount of serum containing media was added to deactivate the trypsin and the cells were concentrated by centrifugation. The supernatant was removed and the cells were resuspended in serum-free medium (SFM). Schwann cells were seeded onto stamped PMMA substrates in SFM for 4-6 hr after which the SFM was removed and replaced with growth media to encourage proliferation. When cells were a confluent monolayer (approximately 3-5 days), they were fixed in 4% formaldehyde (Fisher) for 10 min and rinsed 3 times with PBS. Cells were stored at 4° C. in PBS until stained (no longer than 48 hr). Images of these cells are provided in FIG. 5.

### Fluorescence Labeling

PMMA surfaces with cultured dorsal root ganglia were rinsed with phosphate buffered saline (PBS) and fixed with 4% formaldehyde. The primary antibody was pipetted onto the surface and incubated at room temperature in darkness for 1 hour. Surfaces were then rinsed three times at 15 minutes each with PBS and a secondary fluorescence-conjugated goat antibody was pipetted onto the surface and incubated at room temperature in darkness for 1 hour. The surfaces were imaged on a Zeiss laser scanning confocal microscope for fluorescence at 512 nm. The observed fluorescence was associated with the cells adhered to the PMMA substrate (FIG. 3).

The Schwann cells and laminin patterns were fluorescently stained to contrast the cell body from the laminin pattern. The fixed samples were incubated with primary antibodies (rabbit anti-S100 for Schwann cells, diluted 1:100, DAKO and rat anti-laminin for laminin, diluted 1:100, Sigma) diluted in blocking solution (PBS, 10% goat serum and 0.1% Triton X100 (Sigma)), at room temperature for 1 hr, rinsed 3 times with PBS, incubated with the secondary antibody (Alexafluor 568 conjugated goat-anti-rabbit, diluted 1:100, Molecular Probes, fluorescein-isothiocyanate goat anti-rat, diluted 1:100, Jackson Immunoresearch) diluted in PBS, for 1 hr at room temperature and rinsed 3 times with PBS. The nuclei were stained using ethidium bromide (3.5 μL/7 mL, Molecular Probes) at room temperature for 30 minutes, rinsed 3 times with PBS and covered with an anti-fade preparation.

The samples were imaged on a Zeiss LSM 410 confocal laser-scanning microscope (CLSM) with a computer controlled laser scanning assembly attached to the microscope using a 20× phase contrast objective in fluorescence mode. An Omnichrome 3 line Argon/Krypton laser (488, 568, 647 nm) was used as the excitation source for double fluorescence measurements, with excitation at 488 nm and 568 nm and emission at 512 nm and 605 nm. The images were processed with Zeiss LSM control software.

### Image Analysis

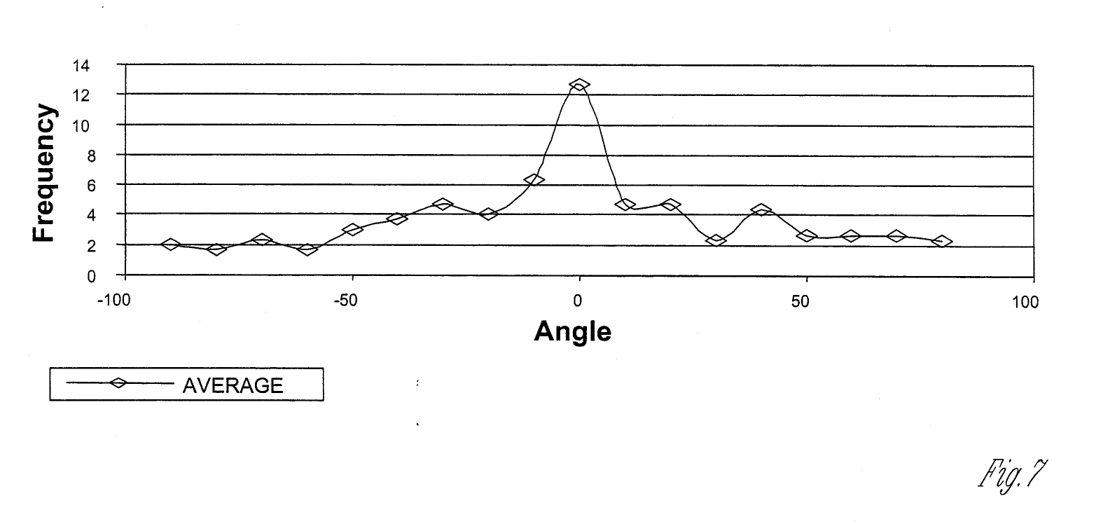

Multiple images were taken randomly per sample and analyzed by Image Pro Plus image analysis software (Media Cybernetics). Using Image Pro Plus, fluorescent images of the stained Schwann cells were converted to black/white, thresholded to separate cells, then cells and cell aggregates were fitted with ellipses (FIG. 6). Major and minor axes were calculated as well as the angle created by the major axis and the direction of patterning (FIG. 7). The data was imported into Microsoft Excel for further manipulation.

The frequencies of orientation angle from the ellipses were separated into 10° increments from 0-180°. The mean angle was determined and converted to 0°, with a new range of +90°, so that distributions could be compared. The direction of laminin patterning was represented by 0° and the frequencies were normalized and plotted.

All publications, patents, and patent documents (including U.S. patent application Ser. Nos. 09/455,861 and 09/508,217; as well as International Patent Application PCT/US98/18816) are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed:

1. A therapeutic device for neural tissue regeneration comprising a tube, or a polymeric sheet that is rolled into a tube, which comprises a biodegradable polymer that biodegrades to provide sustained release of an anti-inflammatory compound to the neural tissue.

2. The device of claim 1 wherein the anti-inflammatory compound is a salicylate.

3. The device of claim 1 wherein the anti-inflammatory compound is a non-steroidal anti-inflammatory compound.

4. The device of claim 1 wherein the anti-inflammatory compound is an aromatic anti-inflammatory compound.

5. The device of claim 1 wherein the anti-inflammatory compound is a cyclooxygenase inhibitor.

6. The device of claim 1 wherein the anti-inflammatory compound is a cyclooxygenase-1 inhibitor.

7. The device of claim 1 wherein the anti-inflammatory compound is a cyclooxygenase-2 inhibitor.

8. The device of claim 1 wherein the anti-inflammatory compound is aceclofenac, acemetacin, E-acetamidocaproic acid, acetaminosalol, acetylsalicylic acid alclofenac, alminoprofen, 3-amino-4-hydroxybutyric acid, amixetrine, ampiroxicam, amtolmetin gaucil, apazone, aspirin, bendazac, benorylate, benoxaprofen, benzpiperylon, benzydamine, bermoprofen, α-bisabolol, bucolome, bucolome, bucloxic acid, bufexamac, bumadizon, butibufen, calcium acetylsalicylate, carprofen, celebrex, choline salicylate, cinmetacin, clopirac, clidanac, diclofenac, difenamizole, difenpiramide, diflunisal, ditazol, droxicam, emorfazone, enfenamic acid, epirizole, etersalate, etodolac, etofenamate, felbinac, fenbufen, fenclozic acid, fenoprofen, fentiazac, fepradinol, feprazone, flunoxaprofen, flurbiprofen, glucametacin, gueiazulene, ibufenac, ibuprofen, ibuproxam, imidazole salicylate, indomethacin, indoprofen, isofezolac, isonixin, isoxepac, isoxicam, ketoprofen, ketorolac, ketorolac tromethamine, lomoxicam, lonazolac, loxoprofen, lysine acetylsalicylate, magnesium salicylate, mefenamic acid, meloxicam, metiazinic acid, mofebutazone, mofezolac, morazone, morpholine salicylate, nabumetone, 1-naphthyl salicylate, naproxen, naproxen sodium, nimesulide, olsalazine, oxaceprol, oxametacine, oxaprozin, oxyphenbutazone, paranyline, parsalmide, perisoxal, phenyl acetylsalicylate phenylbutazone, phenyl salicylate, piroxicam, piketoprofen, pipebuzone, pirazolac, piroxicam, pirprofen, pranoprofen, proglumetacin, propyphenazone, proquazone, protizinic acid, ramifenazone, rofecoxib, S-adenosylmethionine, salacetamide, salsalate, salicylic acid, salicylic acid, sodium salicylate, sulindac, superoxide dismutase, suprofen, suxibuzone, talniflumate, tenidap, tenoxicam, terofenamate, thiazolinobutazone, tiaprofenic acid, tiaramide, tinoridine, tolmetin sodium, tropesin, xenbucin, ximoprofen, zaltoprofen, zileuton, zomepirac or a combination thereof.

9. The device of claim 1 wherein the biodegradable polymer comprises one or more units of formula I:
—R₁-A-L-A-
wherein:
R₁ is a group that will provide an anti-inflammatory agent upon hydrolysis of polymer;
each A is independently an amide linkage, a thioester linkage, or an ester linkage; and
L is a linking group.

10. The device of claim 1 wherein the biodegradable polymer comprises one or more units of formula II:
R₂-A-L-A-R₃-A-L-A-
wherein:
R₂ and R₃ are each independently a group that will yield an anti-inflammatory agent upon hydrolysis of the polymer;
each A is independently an amide, thioester, or ester linkage; and
each L is independently a linking group.

11. The device of claim 12 wherein the anti-inflammatory compound is a salicylate.

12. The device of claim 1 wherein the polymeric sheet has ridges or spacers that are configured to optimally guide the growth and extension of the neural tissue.

13. The device of claim 67 wherein sheet has multiple linear ridges that run the length of the sheet so that upon rolling, the sheet forms a porous tube with a multitude of tubules running from one end of the rolled sheet to another.

14. The device of claim 67 wherein the ridges or spacers are about one micron to about fifty microns in height.

15. The device of claim 67 wherein the length of the polymeric sheet is from about one millimeter to about twenty centimeters.

16. The device of claim 67 wherein the length of the polymeric sheet is from about three millimeters to about ten centimeters.

17. The device of claim 67 wherein the length of the polymeric sheet is from about five millimeters to about five centimeters.

18. The device of claim 1 wherein the tube is of the approximate diameter to accommodate an uninjured neural tissue.

19. The device of claim 1 wherein the tube has a diameter of from about one millimeter to about ten centimeters.

20. The device of claim 1 wherein the tube has a diameter of from about three millimeters to about five centimeters.

21. The device of claim 1 wherein the tube has a diameter of from about five millimeters to about two centimeters.

22. The device of claim 1 further comprising biologically active molecules adsorbed or covalently attached in a pattern on the biodegradable polymer.
